(19) United States
(12) Patent Application Publication
(10) Pub. No.: US 2011/0052602 A1
(43) Pub. Date: Mar. 3, 2011

(54) Diagnosis and Treatment of Age Related Macular Degeneration

(75) Inventors: Josephine Hoh, New Haven, CT (US); Andrew Dewan, New York, NY (US)

(73) Assignee: Yale University, New Haven, CT (US)

(21) Appl. No.: 12/309,616

(22) PCT Filed: Jul. 26, 2007
(86) PCT No.: PCT/US2007/016809

§ 371 Date: Sep. 25, 2009

Related U.S. Application Data
(60) Provisional application No. 60/833,497, filed on Jul. 26, 2006.
(60) Provisional application No. 60/919,409, filed on Mar. 22, 2007.

(51) Int. Cl. A61K 39/395; C12Q 1/68; A61K 31/713; A61K 31/7088; A61P 27/00; C40B 30/04; A61K 38/02

(52) U.S. Cl. 424/172.1; 435/6; 514/44 A; 514/44 R; 506/9; 514/20.8

(57) ABSTRACT

Methods, compositions and kits for diagnosis and treatment of age related macular degeneration.

### CROSS-REFERENCED APPLICATION

This application claims the benefit of U.S. Provisional application No. 60/833,497, filed Jul. 26, 2006 and No. 60/919,409 filed Mar. 22, 2007. The teachings of these referenced provisional applications are incorporated by reference herein in their entirety.

### GOVERNMENT FUNDING

This invention was made with Government support from the National Institutes of Health under Grant No. R01EY15771. The Government has rights in the invention.

### BACKGROUND OF INVENTION

Age related macular degeneration (AMD) is the leading cause of vision loss and blindness among older individuals in the United States and throughout the developed world. It has a complex etiology involving genetic and environmental factors. AMD is broadly classified as either dry (non-neovascular) or wet (neovascular). The dry form is more common, accounting for approximately 85%-90% of patients with AMD, and does not typically result in blindness. The primary clinical sign of dry AMD is the presence of soft drusen with indistinct margins (extracellular protein deposits) between the retinal pigment epithelium (RPE) and Bruch's membrane. The accumulation of these drusen is associated with central geographic atrophy (CGA) and results in blurred central vision. About 10% of AMD patients have the wet form, in which new blood vessels form and break beneath the retina (choroidal neovascularization [CNV]). This leakage causes permanent damage to surrounding retinal tissue, distorting and destroying central vision. Why some individuals develop the more aggressive wet form of AMD, while others have the slowly progressing dry type, is not well understood.

### SUMMARY OF THE INVENTION

The present invention relates to identification of a variation in a human gene correlated with the occurrence of age related macular degeneration, which is useful in identifying or aiding in identifying individuals at risk for developing age related macular degeneration, as well as for diagnosing or aiding in the diagnosis of age related macular degeneration (identifying or aiding in identifying individuals suffering from age related macular degeneration). The methods and compositions are also useful to monitor the status (e.g., progression or reversal) of age related macular degeneration. The methods and compositions of the present invention are useful to identify or aid in identifying individuals of a variety of races and ethnicities and, in particular embodiments, are carried out in order to identify or aid in identifying Caucasian or Asian individuals suffering from or at risk of developing age related macular degeneration. The invention also relates to methods for identifying or aiding in identifying individuals suffering from or at risk for developing age related macular degeneration, methods for diagnosing or aiding in the diagnosis of age related macular degeneration (identifying individuals suffering from/individuals who have age related macular degenerations); polynucleotides (e.g., probes, primers) useful in the methods; diagnostic kits containing probes or primers; methods of treating an individual at risk for or suffering from age related macular degeneration and compositions useful for treating an individual at risk for or suffering from age related macular degeneration.

In one embodiment, the present invention provides polynucleotides for the specific detection of a variant HTRA1 gene that is correlated with the occurrence of age related macular degeneration in humans in a sample from an individual. These polynucleotides are nucleic acid molecules. In specific embodiments, these polynucleotides can be DNA probes that hybridize, under stringent conditions, to a variation in the non-coding region of the human HTRA1 gene (e.g., a variation in the HTRA1 promoter) that is correlated with the occurrence of age related macular degeneration in humans. For example, the probe is one that identifies a variation corresponding to the single nucleotide polymorphism identified as rs11200638. These probes can be from about 8 nucleotides to about 500 nucleotides and in specific embodiments, are from about 10 nucleotides to about 250 nucleotides. In certain embodiments, the polynucleotide probes are about 20 nucleotides (e.g., 15, 16, 17, 18, 19, 20, 21, 22, 23, 24, 25, 26, 27, 28, 29, or 30 nucleotides). In other embodiments, the polynucleotide probes are from about 50 to about 100 nucleotides (e.g., 45, 50, 55, 60, 65, 75, 85, or 100 nucleotides). These probes can contain one or more non-natural or modified nucleotides, including nucleotides that are radioactively, fluorescently, or chemically labeled.

In another embodiment, the polynucleotides are primers that hybridize, under stringent conditions, adjacent to a variation in the non-coding region of the human HTRA1 gene (e.g., a variation in the HTRA1 promoter) that is correlated with the occurrence of age related macular degeneration in humans. In specific embodiments, these primers hybridize immediately adjacent to a variation in the non-coding region of the human HTRA1 gene. In a particular embodiment, a primer hybridizes adjacent to a variation in the HTRA1 promoter, such as the variation described herein that corresponds to the single nucleotide polymorphism identified as rs11200638. Additionally, the present invention provides pairs of polynucleotide primers that detect a variation in the non-coding region of the human HTRA1 gene (e.g., the HTRA1 promoter) that is correlated with the occurrence of age related macular degeneration in humans, wherein the first polynucleotide primer hybridizes to one side of the variation and the second polynucleotide primer hybridizes to the other side of the variation. The pairs of polynucleotide primers hybridize to a region of DNA that comprises a variation in the non-coding region of the human HTRA1 gene (e.g, the promoter region) that is correlated with the occurrence of age related macular degeneration in humans, such as the variation that corresponds to the single nucleotide polymorphism identified as rs11200638. A pair of primers can hybridize in such a manner that the ends of the hybridized primers proximal to the variation are from about 20 to about 10,000 nucleotides apart. For example, hybridization may occur in such a manner that the end of the hybridized primer proximal to the variation is 10, 25, 50, 100, 250, 1000, 5000, or up to 10,000 nucleotides from the variation. In some embodiments, the primers are DNA primers. The primers can be from about 8 nucleotides to about 500 nucleotides. In specific embodiments, the primers can be from about 10 nucleotides to about 250 nucleotides. In certain embodiments, the polynucleotide primers are about 20 nucleotides (e.g., 15, 16, 17, 18, 19, 20, 21, 22, 23, 24, 25, 26, 27, 28, 29, or 30 nucleotides). In other embodiments, the polynucleotide primers are from about 50 to about 100 nucleotides (e.g., 45, 50, 55, 60, 65, 75, 85, or 100 nucleotides).These primers can contain one or more non-natural or modified nucleotides, including nucleotides that are radioactively, fluorescently, or chemically labeled.

In one embodiment, the invention relates to a method of identifying or aiding in identifying an individual suffering from or at risk of developing age related macular degeneration, comprising determining whether a sample obtained from the individual comprises a variant HTRA1 that is correlated with age related macular degeneration, such as a variation in a non-coding region (e.g., a promoter). In a specific embodiment, the variation in the promoter corresponds to the single nucleotide polymorphism identified as rs11200638. The methods of this invention can comprise, in addition to determining whether an HTRA1 variant is present, determining whether one or more additional variants that are correlated with the occurrence of age related macular degeneration is present in an individual being assessed. Additional variants, other than an HTRA1 variant, that can be detected include, but are not limited to, a variation in nucleic acids (DNA, RNA) encoding the CFH protein (e.g., a variation encoding histidine at position 402 of the CFH protein); a variation encoding an amino acid residue other than alanine at position 69 of the protein LOC387715 (e.g., a serine at that position); and a variation corresponding to the single nucleotide polymorphism identified as rs10490924. Some or all of these variants, as well as others correlated with the occurrence of age related macular degeneration, can be determined and/or quantified in a sample from an individual being assessed.

In a further embodiment, the invention relates to a method of monitoring the status of age related macular degeneration in an individual (human). The method is useful to assess, for example, whether age related macular degeneration has progressed (reached a more advanced or later stage) in an individual. This is useful, for example, in assessing the effects/effectiveness of treatments an individual has received. The methods of this invention can help show, for example, that a treatment has been effective, in that it can show if regression (amelioration, partial or complete) of AMD has occurred. The method can also be used to assess whether AMD in an individual has progressed (worsened). This embodiment can be carried out, for example, by assessing the extent to which a variant HTRA1 gene comprising a variation in a noncoding region as described herein is present in a sample obtained from the individual. If the variant HTRA1 is present in the sample to a lesser extent following treatment (than prior to treatment), this is an indication of regression of AMD and that treatment was effective.

In one embodiment the present invention relates to a method of detecting, in a sample obtained from an individual, a variant HTRA1 gene, such as a variant HTRA1 gene that comprises a variation in a non-coding region (e.g., the promoter) that is correlated with the occurrence of age related macular degeneration in humans. The method comprises: (a) combining the sample obtained from the individual (human) with a polynucleotide probe that hybridizes, under stringent conditions, to a variation in the non-coding region of the human HTRA1 gene that is correlated with the occurrence of age related macular degeneration in humans, but not to the corresponding region of a wildtype HTRA1 gene; and (b) determining whether hybridization occurs, wherein the occurrence of hybridization indicates that a variant HTRA1 gene that is correlated with the occurrence of age related macular degeneration is present in the sample. In a specific embodiment, the polynucleotide probe hybridizes, under stringent conditions, to a variation in the HTRA1 promoter (such as the variation that corresponds to the single nucleotide polymorphism identified as rs11200638), but not to the wildtype HTRA1 promoter and if hybridization occurs, it is an indication that an HTRA1 promoter that includes a variation that is correlated with AMD is present in the sample.

In another embodiment, the present invention relates to a method of detecting, in a sample obtained from an individual (human), a variant HTRA1 gene that is correlated with the occurrence of age related macular degeneration in humans, wherein the method comprises: (a) combining the sample obtained from the individual (sample) with a polynucleotide probe that hybridizes, under stringent conditions, to a variation in the non-coding region of the human HTRA1 gene that is correlated with the occurrence of age related macular degeneration in humans, thereby producing a combination (test combination); (b) maintaining the combination produced in step (a) under stringent hybridization conditions; and (c) comparing hybridization that occurs in the test combination with hybridization in a control. The control is the same as (a) and (b) above, except that the polynucleotide probe (control probe) does not bind to a variation in the non-coding region of the human HTRA1 gene (e.g., does not bind to a variation in the HTRA1 promoter) that is correlated with the occurrence of age related macular degeneration in humans, or binds only to a wildtype HTRA1 gene. The sample used in the control is the same type of sample as used in (a). The combination produced by combining the sample with a control probe is referred to as a control combination. The test combination and the control combination are treated the same (subjected to substantially the same conditions). The occurrence of hybridization in the test combination, but not in the control combination, indicates that a variant HTRA1 gene that correlates with age related macular degeneration is present in the sample. For example, hybridization in the test combination that includes a polynucleotide probe that hybridizes to a variation in the HTRA1 promoter that is correlated with the occurrence of AMD indicates that a variant gene in which there is a variation in the HTRA1 promoter is present in the sample. In a specific embodiment, the extent of hybridization is determined in step (c). In a specific embodiment, the variation in the HTRA1 promoter is the variation that corresponds to the single nucleotide polymorphism identified as rs11200638.

In yet another embodiment, the present invention relates to a method of detecting, in a sample obtained from an individual (sample), a variant HTRA1 gene that is correlated with the occurrence of age related macular degeneration in humans, wherein the method comprises: (a) combining a first portion of the sample with a polynucleotide probe that hybridizes, under stringent conditions, to a variation in the non-coding region of the HTRA1 gene that is correlated with the occurrence of age related macular degeneration in humans; (b) combining a second portion of the sample with a polynucleotide probe that hybridizes, under stringent conditions, to a wildtype HTRA1 gene; and (c) determining whether hybridization occurs, wherein the occurrence of hybridization in the first portion, but not in the second portion, indicates that a variant HTRA1 gene that is correlated with the occurrence of age related macular degeneration is present in the sample. In a specific embodiment, the variant HTRA1 gene comprises a variation in the HTRA1 promoter, such as the variation that corresponds to the single nucleotide polymorphism identified as rs11200638.

In specific embodiments, the polynucleotide probe used in the methods described above is a DNA probe. In specific embodiments, the polynucleotide probe is from about 8 nucleotides to about 500 nucleotides.

In certain embodiments, the methods described further comprise combining the sample with a second probe that hybridizes, under stringent conditions, to a variation in a gene, other than the HRTA1 variant (such as a variant that comprises a variation in the HTRA1 promoter), that is correlated with the occurrence of age related macular degeneration in humans. For example, the second probe detects a variation in DNA encoding a variation in the CFH protein that is correlated with age related macular degeneration. In specific embodiments, the second probe detects a variation encoding histidine at position 402 of the CFH protein. In another embodiment, the second probe detects a variation encoding an amino acid residue other than alanine at position 69 of the protein LOC387715, such as a variation encoding serine at position 69. In yet another embodiment, the second probe detects the variation corresponding to the single nucleotide polymorphism identified as rs10490924. Some or all of these variants, as well as others correlated with the occurrence of age related macular degeneration, can be determined and/or quantified, in conjunction with a variant of HTRA1, in a sample from an individual being assessed.

In another embodiment, the present invention relates to a method of detecting, in a sample obtained from an individual, a variant HTRA1 gene (e.g., a variation in a non-coding region, such as a variation in the human HTRA1 promoter) that is correlated with the occurrence of age related macular degeneration in humans, wherein the method comprises: (a) combining the sample with a pair of polynucleotide primers, wherein the first polynucleotide primer hybridizes to one side of position −512 from the putative transcription start site of the human HTRA1 gene in humans and the second polynucleotide primer hybridizes to the other side of position −512 from the putative transcription start site of the HTRA1 gene in humans; (b) amplifying DNA in the sample, thereby producing amplified DNA; (c) sequencing amplified DNA; and (d) detecting in the DNA a variation of the wild-type sequence of the promoter region of the HTRA1 gene, wherein detection of the variation in amplified DNA indicates that a variant HTRA1 gene that is correlated with the occurrence of age related macular degeneration in humans is present in the sample. DNA can be amplified using methods known to those in the art, such as the polymerase chain reaction (PCR).

In a specific embodiment of the method described, a second set of primers that hybridize to either side of a variation in a gene, other than an HTRA1 variant, that is correlated with the occurrence of age related macular degeneration in humans, is used. For example, the second probe detects a variation in DNA encoding a variation in the CFH protein that is correlated with age related macular degeneration. In specific embodiments, the variation detected by the second set of primers is the variation encoding histidine at position 402 of the CFH protein, corresponding to SNP rs 1061170. In another embodiment, the variation detected by the second set of primers is a variation encoding an amino acid residue other than alanine at position 69 of the protein LOC387715, such as serine at position 69. In yet another embodiment, the variation detected by the second set of primers is the variation corresponding to the single nucleotide polymorphism identified as rs10490924.

In a further embodiment, the present invention relates to a method of identifying or aiding in identifying an individual suffering from or at risk for developing age related macular degeneration which comprises assaying a sample obtained from the individual for the presence of a variant HTRA1 gene that is correlated with the occurrence of age related macular degeneration in humans, wherein the presence of a variant HTRA1 gene in the sample indicates that the individual has or is at risk for developing age related macular degeneration. In a specific embodiment, such a method of identifying or aiding in identifying comprises assaying a sample obtained from the individual for the presence of a variation in a non-coding region of HTRA1, such as a variation in the HTRA1 promoter, that is correlated with the occurrence of age related macular degeneration. The presence of such a variation (e.g., a variation in HTRA1, promoter, such as single nucleotide polymorphism [G→A] at position −512 from the putative transcription start site of the promoter of the human HTRA1 gene) indicates that the individual has or is at risk of developing age related macular degeneration.

In another embodiment, the present invention relates to a method of identifying or aiding in identifying an individual suffering from or at risk for developing age related macular degeneration, comprising: (a) combining a sample obtained from the individual with a polynucleotide probe that hybridizes, under stringent conditions, to a variation in the non-coding region of the human HTRA1 gene (e.g., a variation in the HTRA1 promoter) that is correlated with the occurrence of age related macular degeneration in humans, but not to the corresponding region of a wild-type HTRA1 gene; and (b) determining whether hybridization occurs, wherein the occurrence of hybridization indicates that the individual is at risk for developing age related macular degeneration. In a specific embodiment, the variation in the HTRA1 promoter corresponds to the single ncleotide polymorphism identified as rs11200638.

In a specific embodiment of the method described above, a second probe that hybridizes, under stringent conditions, to a variation, other than the HTRA1 variant, that is correlated with the occurrence of age related macular degeneration in humans is used. For example, the second probe detects a variation in DNA encoding the CFH protein. In specific embodiments, the variation detected by the second probe is a variation encoding histidine at position 402 of the CFH protein, corresponding to SNP rs 1061170. In another embodiment, the variation detected by the second probe is a variation encoding an amino acid residue other than alanine at position 69 of the protein LOC387715, such as serine at position 69. In yet another embodiment, the variation detected by the second probe is the variation corresponding to the single nucleotide polymorphism identified as rs10490924.

In yet another embodiment, the present invention relates to a method of identifying or aiding in identifying an individual suffering from or at risk for developing age related macular degeneration comprising: (a) obtaining DNA from an individual; (b) sequencing a region of the DNA that comprises the nucleotide at position −512 from the putative transcription start site of the HTRA1 gene in humans; and (c) determining whether a variation of the wild-type sequence of the promoter region of the HTRA1 gene is present in the DNA, wherein if the variation is present in the DNA, the individual has or is at risk for developing age related macular degeneration.

In a specific embodiment, the method described above comprises additionally sequencing a second variant, other than an HTRA1 variant, that is correlated with the occurrence of age related macular degeneration in humans. For example, the second variant can be a variation in DNA encoding the CFH protein that is correlated with age related macular degeneration in humans.

In specific embodiments, the second variation is a variation encoding histidine at position 402 of the CFH protein, corresponding to SNP rs 1061170. In another embodiment the second variation is a variation encoding an amino acid residue other than alanine at position 69 of the protein LOC387715, such as serine at position 69. In yet another embodiment,the second variation is a variation corresponding to the single nucleotide polymorphism identified as rs10490924.

A diagnostic method of this invention can comprise, in addition to detecting the variation in the human HTRA1 gene identified as SNP rs 1120063, detecting additional variations that are correlated with the risk of developing AMD, such as variations in the human CFH gene, identified as SNP rs 1061170, or variations in the human gene locus LOC3 87715, identified as SNP rs 10490924. Such a diagnostic test may make it possible to predict the severity (the extent of progression) of AMD based on the information obtained from the test and by knowledge about a patient's habits (e.g., potentially additional risk factors, such as smoking and obesity).

In another embodiment, the present invention relates to a diagnostic kit for detecting a variant HTRA1 gene (e.g., a variant gene having a variation in a non-coding region, such as a variation in the HTRA1 promoter) in a sample from an individual. The diagnostic kit comprises: (a) at least one container means having disposed therein at least one polynucleotide probe that hybridizes, under stringent conditions, to a variation in the non-coding region of the HTRA1 gene (such as a variation in the HTRA1 promoter) that is correlated with the occurrence of age related macular degeneration in humans; and (b) a label and/or instructions for the use of the diagnostic kit in the detection of a variant HTRA1 gene in a sample.

In a specific embodiment, the kit described above additionally comprises at least one additional probe (a second probe) that hybridizes, under stringent conditions, to a variation, other than the HTRA1 variant, that is correlated with the occurrence of age related macular degeneration in humans. The variant can be, for example, a variation in DNA encoding the CFH protein. In specific embodiments, the variation detected by the second probe is a variation encoding histidine at position 402 of the CFH protein, corresponding to SNP rs 1061170. In another embodiment, the variation detected by the second probe is a variation encoding an amino acid residue other than alanine at position 69 of the protein LOC387715, such as serine at position 69. In yet another embodiment the variation detected by the second probe is the variation corresponding to the single nucleotide polymorphism identified as rs10490924.

In yet another embodiment, the present invention is a diagnostic kit for detecting a variant HTRA1 gene (e.g., a variant gene having a variation in a non-coding region, such as a variation in the HTRA1 promoter) in a sample from an individual, comprising: (a) at least one container means having disposed therein at least one polynucleotide primer that hybridizes, under stringent conditions, adjacent to one side of a variation in the non-coding region of the HTRA1 gene (e.g., a variant gene having a variation in the HTRA1 promoter) that is correlated with the occurrence of age related macular degeneration in humans; and (b) a label and/or instructions for the use of the diagnostic kit in the detection of HTRA1 in a sample.

In a specific embodiment, the kit described above additionally comprises at least a second polynucleotide primer that hybridizes, under stringent conditions, to the other side of the variation in the non-coding region of the HTRA1 gene that is correlated with the occurrence of age related macular degeneration in humans.

In another specific embodiment, the kit described above additionally comprises a second set of primers that hybridize to either side of a variation, other than the HTRA1 variant, that is correlated with the occurrence of age related macular degeneration in humans.

In specific embodiments, the variation detected by the second set of primers is a variation encoding histidine at position 402 of the CFH protein, corresponding to SNP rs 1061170. In another embodiment, the variation detected by the second set of primers is a variation encoding an amino acid residue other than alanine at position 69 of the protein LOC387715, such as serine at position 69. In yet another embodiment, the variation detected by the second set of primers is a variation corresponding to the single nucleotide polymorphism identified as rs10490924.

In another embodiment, the present invention relates to a composition for treating an individual subject suffering from or at risk for developing age related macular degeneration that comprises: (a) an effective amount of an inhibitor of HTRA1 activity and (b) a pharmaceutically acceptable carrier.

In another embodiment, the present invention relates to a composition for treating a subject suffering from or at risk for developing age related macular degeneration comprising: (a) a nucleic acid molecule comprising an antisense sequence that hybridizes to HTRA1 gene or mRNA; and (b) a pharmaceutically acceptable carrier. In a specific embodiment, hybridization of the antisense sequence to the HTRA1 gene reduces the extent to which RNA is transcribed from the HTRA1 gene. Hybridization of the antisense sequence to the HTRA1 mRNA reduces the amount of protein translated from the HTRA1 mRNA and/or alters the splicing of the HTRA1 mRNA. In a specific embodiment, the invention provides nucleic acid molecules that include one or more modified nucleotides or nucleosides that enhance in vivo stability, transport across the cell membrane, or hybridization to a HTRA1 gene or mRNA.

In another embodiment, the present invention provides a composition for treating a subject suffering from or at risk for developing age related macular degeneration comprising: (a) a nucleic acid molecule comprising a siRNA or miRNA sequence, or a precursor thereof, that hybridizes to HTRA1 gene or mRNA and (b) a pharmaceutically acceptable carrier.

Hybridization of the siRNA or miRNA sequence to the HTRA1 gene reduces the extent to which RNA is transcribed from the HTRA1 gene. Hybridization of the siRNA or miRNA sequence to the HTRA1 mRNA reduces the amount of protein translated from the HTRA1 mRNA, and/or alters the splicing of the HTRA1 mRNA.

In a specific embodiment, the invention relates to nucleic acid molecules that include one or more modified nucleotides or nucleosides that enhance in vivo stability, transport across the cell membrane, or hybridization to a HTRA1 gene or mRNA.

In another embodiment, the present invention relates to a composition for treating a subject suffering from or at risk for developing age related macular degeneration, comprising: (a) an aptamer that binds to the HTRA1 polypeptide; and (b) a pharmaceutically acceptable carrier. Binding of the aptamer to the HTRA1 polypeptide reduces the activity of the HTRA1 polypeptide.

In another embodiment, the present invention relates to a composition for treating a subject suffering from or at risk for developing age related macular degeneration, comprising (a) a small molecule that binds to the HTRA1 polypeptide; and (b) a pharmaceutically acceptable carrier. Binding of the small molecule to the HTRA1 polypeptide reduces the activity of the HTRA1 polypeptide.

In another embodiment, the present invention relates to a composition for treating a subject suffering from or at risk for developing age related macular degeneration, comprising: (a) an antibody that binds to the HTRA1 polypeptide; and (b) a pharmaceutically acceptable carrier. Binding of the antibody to the HTRA1 polypeptide reduces the activity of the HTRA1 polypeptide.

In another embodiment, the present invention relates to a composition for treating a subject suffering from or at risk for developing age related macular degeneration, comprising: (a) a dominant negative variant of the HTRA1 polypeptide that competes with the wildtype HTRA1 polypeptide; and (b) a pharmaceutically acceptable carrier. Binding of the antibody to the HTRA1 polypeptide reduces the activity of the HTRA1 polypeptide.

In another embodiment, the present invention relates to a composition for treating a subject suffering from or at risk for developing age related macular degeneration, at least containing the following: (a) an agent that alters the levels of a transcription factor that binds to the promoter of the HTRA1 gene; and (b) a pharmaceutically acceptable carrier.

In a specific embodiment, the agent is (a) an over-expression vector, antisense RNA, siRNA, miRNA, aptamer, small molecule, or antibody directed to a transcription factor or (b) a dominant negative variant of the transcription factor. For example, the over-expression vector, antisense RNA, siRNA, miRNA, aptamer, small molecule, or antibody is directed to the transcription factor SRF or AP2 alpha. the dominant negative variant is, for example, a dominant negative variant of SRF or AP2 alpha.

In another embodiment, the present invention relates to a composition for treating a subject suffering from or at risk for developing age related macular degeneration, comprising: (a) an agent that inhibits secretion of the HTRA1 polypeptide; and (b) a pharmaceutically acceptable carrier.

In a specific embodiment, the agent that inhibits its secretion of HTRA1 is a dominant negative variant of the HTRA1 polypeptide that competes with wildtype HTRA1 for secretion. In another specific embodiment, the agent is an aptamer, small molecule, or antibody that is directed to the HTRA1 polypeptide.

In another embodiment, the present invention is a method of treating an individual (human) suffering from or at risk for developing age related macular degeneration, wherein an effective amount of any of the compositions described herein is administered to the individual.

### BRIEF DESCRIPTION OF THE DRAWINGS

FIG. 1 is a schematic depicting the genes in the 4-gamete region on chromosome 10q26, as well as the location of SNPs genotyped by microarrays (+) and identified through sequencing (I). SNP rs10490924 is labeled as “8” and rs11200638 is marked with an asterisk.

FIGS. 2A-B are graphs depicting results obtained by quantitative PCR (qPCR) of ChIP DNA prepared from HeLaS3 cells: FIG. 2A AP-2α (solid line), FIG. 2B SRF (solid line), and (FIGS. 2A and 2B) normal rabbit immunoglobulin G (dashed line) represent the immunoprecipitations analyzed. Positive and negative control promoters were also tested. The log(ΔRn) (y-axis) is plotted against the PCR cycle number (x-axis). The ΔΔCt values fold increase of transcription calculated relative to reference PCR reactions) are shown in parentheses.

FIG. 3 is a depiction of a computation analysis of the HTRA1 promoter sequence. Human and Mouse orthologous sequences in the HTRA1 promoter; the conserved nucleotides are marked with *.

FIG. 4A is a plot depicting log P-values (y axis) from association analyses for the 15 SNPs at the 10q AMD region using 442 AMD cases and 309 controls (see also table 2). For the rs10490924 (square) and rs11200638 (triangle) SNPs, associations were derived from a larger sample size (581 AMD cases).

FIG. 4B is a bar graph depicting results obtained by Real-time RT-PCR semi-quantitative analysis of HTRA1 RNA levels in blood lymphocytes from three AMD patients with the AA genotype and three normal controls with the GG genotype. The statistical significance of the differences in expression level was examined using an independent samples t test (SPSS version 13.0): AA:GG (P=0.02). The error bars indicate the 95.0% confidence interval of the mean. 1577275.1

### DETAILED DESCRIPTION OF THE INVENTION

The discovery that a variation in the non-coding region of the HTRA1 gene is associated with AMD is useful for the diagnosis and treatment of individuals, such as those suffering from or at risk of developing age related macular degeneration. The determination of the genetic constitution of the HTRA1 gene in an individual is useful as the basis for diagnosing or treating AMD at earlier stages, or even before an individual displays symptoms of AMD. Furthermore, diagnostic tests to genotype HTRA1 may allow individuals to alter their behavior to minimize environmental risks to AMD (e.g., smoking, obesity). The present invention relates to the identification of a variant HTRA1 gene correlated with the occurrence of AMD, which is useful in identifying or aiding in identifying individuals at risk for developing AMD, as well as for diagnosing or aiding in the diagnosis of AMD. It also relates to methods for identifying or aiding in identifying individuals at risk for developing AMD, methods for diagnosing or aiding in the diagnosis of AMD, methods for monitoring the status (e.g., progression, reversal) of AMD, polynucleotides (e.g., probes, primers) useful in the methods, diagnostic kits containing probes or primers, methods of treating an individual at risk for or suffering from AMD and compositions useful for treating an individual at risk for or suffering from AMD.

Applicants have shown that a common variation in the non-coding region of the human HTRA1 gene is strongly associated with AMD. The present invention relates to methods and compositions for detecting such variations that are correlated with the occurrence of age related macular degeneration in humans.

HtrA (high temperature requirement) was initially identified in E. coli as a heat shock protein and was subsequently found to exist ubiquitously in microbes, plants and animals. Human HTRA1 is a member of the HtrA family of serine proteases. Its structural features include a highly conserved trypsin-like serine protease domain, as well as an insulin-like growth factor binding protein domain and a Kazal-type serine protease inhibitor motif.

Down regulation of human HTRA1 gene expression has been observed in certain cancers (ovarian cancer, melanoma), in close correlation with malignant progression and metastasis of these tumors. Overexpression of HTRA1 in tumors on the other hand suppresses proliferation and migration of tumor cells, suggesting that HTRA1 has tumor suppressive properties in certain cancers. In contrast to tumor tissue, HTRA1 expression is upregulated in skeletal muscle of Duchenne muscular dystrophy and in cartilage of osteoarthitic joints, which may contribute to the development of this disease.

The variation in the non-coding region of the human HTRA1 gene that is strongly associated with AMD described herein is the variation that corresponds to the single nucleotide polymorphism identified as rs 11200638. The variation is a single nucleotide polymorphism (G→A) in the promoter region of the human HTRA1 gene. A single nucleotide polymorphism located in the non-coding regulating region at position −512 relative to the putative transcription start site of the human HTRA1 gene on human chromosome 10 is associated with the risk of developing age related macular degeneration (AMD). This single nucleotide polymorphism (SNP) is identified as rs 11200638. Based on this association as disclosed herein, it is possible to determine whether an individual is at risk of developing AMD using diagnostic tests that can be conducted routinely and reproducibly on a variety of samples from the individual. If the HTRA1 variant is detected in a sample using a diagnostic test, this finding can be used to determine whether an individual is at risk of developing AMD, aid in diagnosing AMD or confirm an AMD diagnosis based on other data.

The promoter region is the regulatory region of a gene that is a non-coding coding section that is not translated into a protein sequence. Certain cellular transcription factors can bind to the promoter region of a gene to influence its transcriptional activity. The single nucleotide polymorphism that is identified as rs11200638 is located at position −512 relative to the putative transcription start site.

Polymorphisms in the promoter region can, under certain circumstances, alter the ability of transcription factors to bind to the promoter, for example by changing the affinity of a transcription factor binding site located within the promoter sequence, for the corresponding transcription factor.

Changes in transcription factor binding to the promoter can affect the activity of a promoter, for example transcriptional activity of the promoter, which can influence the rate of transcription of a gene. A higher rate of transcription can lead to more corresponding protein to be made. A lower rate of transcription can lead to less corresponding protein being made. Changes in protein levels can affect many biological processes and can potentially have debilitating effects.

The term “G→A”, as used herein, means a change of a single nucleotide base from a wildtype G to a variant A at a certain position within the genome. Such changes are known in the art as “single nucleotide polymorpisms” or SNP(s). Such changes can affect one or both alleles and this can occur in a heterozygoes or homozygoes manner. The “G→A” change, as used herein, is understood to include both possible genotypes; a heterozygoes G→A change and a homozygoes GG→AA change, even though it is herein referred to only as G→A for reasons of simplicity.

An HTRA1 gene can be the cDNA or the genomic form of the gene, which may include upstream and downstream regulatory sequences, such as promoter sequences. The HTRA1 polypeptide can be encoded by a full length coding sequence or by any portion of the coding sequence, so long as the desired activity or functional properties (e.g., enzymatic activity, ligand binding, signal transduction, etc.) of the full-length or fragment are retained. The HTRA1 gene may further include sequences located adjacent to the coding region on both the 5′ and 3′ ends for a distance of about 1-2 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

To provide an overall understanding of the invention, certain illustrative embodiments will now be described, including compositions and methods for identifying or aiding in identifying individuals at risk for developing AMD, as well as for diagnosing or aiding in the diagnosis of AMD. However, it will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

HTRA1 polynucleotide probes and primers

In certain embodiments, the invention relates to isolated and/or recombinant polynucleotides that specifically detect a variation in the non-coding region of the HTRA1 gene that is correlated with the occurrence of AMD. Such as a variation in the HTRA1 promoter is the single nucleotide polymorphism that is identified as rs 11200638. Polynucleotide probes of the invention hybridize to such a variation (referred to as a variation of interest) in a HTRA1 gene in a specific manner and typically have a sequence which is fully or partially complementary to the sequence of the variation. Polynucleotide probes can also hybridize to sequences on either or both sides of the variation of interest; they can hybridize to flanking sequences on either or both sides of the variation of interest. Polynucleotide probes of the invention can hybridize to a segment of target DNA such that the variation aligns with a central position of the probe, or the variation may align with another position, such as a terminal position, of the probe.

In one embodiment, a polynucleotide probe of the invention hybridizes, under stringent conditions, to a nucleic acid molecule comprising a variant HTRA1 gene, or a portion or allelic variant thereof, that is correlated with the occurrence of AMD in humans. For example, a polynucleotide probe hybridizes to a variation in the HTRA1 promoter that is correlated with the occurrence of AMD in humans in a specific example the variation is a nucleotide base other than G at position −512 relative to the putative transcription sart site of the human HTRA1 gene. A polynucleotide probe of the invention hybridizes, under stringent conditions, to a nucleic acid molecule (e.g., DNA) of a HTRA1 gene, or an allelic variant thereof, wherein the nucleic acid molecule comprises a variation that is correlated with the occurrence of AMD in humans, such as the variations that is identified as SNP rs 11200638.

In certain embodiments, a polynucleotide probe of the invention is an allele-specific probe. The design and use of allele-specific probes for analyzing polymorphisms is described by, e.g., Saiki et al., Nature 324:163-166 (1986); Dattagupta, EP 235726; and Saiki WO 89/11548. Allele-specific probes can be designed to hybridize to a segment of a target DNA from one individual and not to hybridize to the corresponding segment from another individual due to the presence of different polymorphic forms or variations in the respective segments from the two individuals. Hybridization conditions should be sufficiently stringent that there is a significant difference in hybridization intensity between alleles. In some embodiments, a probe hybridizes to only one of the alleles.

A variety of variations in the HTRA1 gene that predispose an individual to AMD can be detected by the methods and polynucleotides described herein. In a specific embodiment the variation in the HTRA1 gene that is correlated with the occurrence of AMD is a variation in the non-coding region of the HTRA1 gene. More specifically the variation is a single nucleotide polymorphism (G→A) at position −512 from the putative transcription start site of the promoter of the human HTRA1 gene. This polymorphism is identified as rs11200638. Polymorphisms other than that at position −512, described above, can be detected in the non-coding region of the human HTRA1 gene particularly within the promoter sequence using the methods and polynucleotides described herein.

In another embodiment, any nucleotide polymorphism of a coding region, exon, exon-intron boundary, signal peptide, 5-prime untranslated region, promoter region, enhancer sequence, 3-prime untranslated region or intron that is associated with AMD can be detected. These polymorphisms include, but are not limited to, changes that: alter the amino acid sequence of the proteins encoded by the HTRA1 gene, produce alternative splice products, create truncated products, introduce a premature stop codon, introduce a cryptic exon, alter the degree or expression to a greater or lesser extent, alter tissue specificity of HTRA1 expression, introduce changes in the tertiary structure of the proteins encoded by HTRA1, introduce changes in the binding affinity or specificity of the proteins expressed by HTRA1 or alter the function of the proteins encoded by HTRA1.

The subject polynucleotides are further understood to include polynucleotides that are variants of the polynucleotides described herein, provided that the variant polynucleotides maintain their ability to specifically detect a variation in the non-coding region of the HTRA1 gene, such as a variation in the HTRA1 promoter (e.g., a variation that encodes a change of position −512 from the putative transcription start site or the human HTRA1 gene) that is correlated with the occurrence of AMD. Variant polynucleotides may include, for example, sequences that differ by one or more nucleotide substitutions, additions or deletions.

In certain embodiments, the isolated polynucleotide is a probe that hybridizes, under stringent conditions, to a variation in the non-coding region of the HTRA1 gene that is correlated with the occurrence of AMD in humans. In one embodiment, the probe hybridized to a variation that is the single nucleotide polymorphism (G→A) at position −512 from the putative transcription start site of the promoter of the human HTRA1 gene, which is identified as rs11200638. As used herein, the term “hybridization” is used in reference to the pairing of complementary nucleic acids. The term “specifically detects” as used in reference to a polynucleotide is intended to mean, as is generally understood in the art, that the polynucleotide is selective between a nucleic acid of interest and other nucleic acids not of interest. Such a polynucleotide can distinguish between the sequence of a nucleic acid of interest and the sequence of a nucleic acid that is not interest such that the polynucleotide is useful for, at minimum, detecting the presence of the nucleic acid sequence of interest in a particular type of biological sample. The term “probe” refers to a polynucleotide that is capable of hybridizing to a nucleic acid of interest. The polynucleotide may be naturally occurring, as in a purified restriction digest, or it may be produced synthetically, recombinantly or by nucleic acid amplification (e.g., PCR amplification).

It is well known in the art how to perform hybridization experiments with nucleic acid molecules. The skilled artisan is familiar with the hybridization conditions required in the present invention and understands readily that appropriate stringency conditions which promote DNA hybridization can be varied. Such hybridization conditions are referred to in standard text books, such as Molecular Cloning: A Laboratory Manual, Cold Spring Harbor Laboratory (2001); and Current Protocols in Molecular Biology, eds. Ausubel et al., John Wiley & Sons (1992). Particularly useful in methods of the present invention are polynucleotides which are capable of hybridizing to a variant HTRA1 gene, or a region of a variant HTRA1 gene, under stringent conditions. Under stringent conditions, a polynucleotide that hybridizes to a variant HTRA1 gene does not hybridize to a wildtype HTRA1 gene.

Nucleic acid hybridization is affected by such conditions as salt concentration, temperature, organic solvents, base composition, length of the complementary strands, and the number of nucleotide base mismatches between the hybridizing nucleic acids, as will readily be appreciated by those skilled in the art. Stringent temperature conditions will generally include temperatures in excess of 30° C., or may be in excess of 37° C. or 45° C. Stringency increases with temperature. For example, temperatures greater than 45° C. are highly stringent conditions. Stringent salt conditions will ordinarily be less than 1000 mM, or may be less than 500 mM or 200 mM. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. Particularly useful in methods of the present invention are polynucleotides which are capable of hybridizing to a variant HTRA1 gene, or a region of a variant HTRA1 gene, under stringent conditions. It is understood, however, that the appropriate stringency conditions may be varied in the present invention to promote DNA hybridization. In certain embodiments, polynucleotides of the present invention hybridize to a variant HTRA1 gene, or a region of a variant HTRA1 gene, under highly stringent conditions. Under stringent conditions, a polynucleotide that hybridizes to a variation in the non-coding region of the HTRA1 gene does not hybridize to a wildtype HTRA1 gene. In one embodiment, the invention provides nucleic acids which hybridize under low stringency conditions of 6.0×SSC at room temperature followed by a wash at 2.0×SSC at room temperature. The combination of parameters, however, is much more important than the measure of any single parameter. See, e.g., Wetmur and Davidson, 1968. Probe sequences may also hybridize specifically to duplex DNA under certain conditions to form triplex or higher order DNA complexes. The preparation of such probes and suitable hybridization conditions are well known in the art. One method for obtaining DNA encoding the biosynthetic constructs disclosed herein is by assembly of synthetic oligonucleotides produced in a conventional, automated, oligonucleotide synthesizer.

A polynucleotide probe or primer of the present invention may be labeled so that it is detectable in a variety of detection systems, including, but not limited, to enzyme (e.g., ELISA, as well as enzyme-based histochemical assays), fluorescent, radioactive, chemical, and luminescent systems. A polynucleotide probe or primer of the present invention may further include a quencher moiety that, when placed in proximity to a label (e.g., a fluorescent label), causes there to be little or no signal from the label. Detection of the label may be performed by direct or indirect means (e.g., via a biotin/avidin or a biotin/stretpavidin linkage). It is not intended that the present invention be limited to any particular detection system or label.

In another embodiment, the isolated polynucleotide of the invention is a primer that hybridizes, under stringent conditions, adjacent, upstream, or downstream to a variation in the non-coding region of the HTRA1 gene that is correlated with the occurrence of AMD in humans. The isolated polynucleotide may hybridize, under stringent conditions, to a nucleic acid molecule comprising all or a portion of a variant HTRA1 gene that is correlated with the occurrence of AMD in humans. Alternatively, the isolated polynucleotide primer may hybridize, under stringent conditions, to a nucleic acid molecule comprising at least 50 contiguous nucleotides of a variant HTRA1 gene that is correlated with the occurrence of AMD in humans. For example, a polynucleotide primer of the invention can hybridize adjacent, upstream, or downstream to the region of the human HTRA1 gene that encodes a change at position −512 from the putative transcription start site of the promoter of the human HTRA1 gene, which is identified as rs 11200638.

As used herein, the term “primer” refers to a polynucleotide that is capable of acting as a point of initiation of nucleic acid synthesis when placed under conditions in which synthesis of a primer extension product that is complementary to a nucleic acid strand occurs (for example, in the presence of nucleotides, an inducing agent such as DNA polymerase, and suitable temperature, pH, and electrolyte concentration). Alternatively, the primer may be capable of ligating to a proximal nucleic acid when placed under conditions in which ligation of two unlinked nucleic acids occurs (for example, in the presence of a proximal nucleic acid, an inducing agent such as DNA ligase, and suitable temperature, pH, and electrolyte concentration). A polynucleotide primer of the invention may be naturally occurring, as in a purified restriction digest, or may be produced synthetically. The primer is preferably single stranded for maximum efficiency in amplification, but may alternatively be double stranded. If double stranded, the primer is first treated to separate its strands before being used. Preferably, the primer is an oligodeoxyribonucleotide. The exact lengths of the primers will depend on many factors, including temperature, source of primer and the use of the method. In certain embodiments, the polynucleotide primer of the invention is at least 10 nucleotides long and hybridizes to one side or another of a variation in the non-coding region of the HTRA1 gene that is correlated with the occurrence of AMD in humans. The subject polynucleotides may contain alterations, such as one or more nucleotide substitutions, additions or deletions, provided they hybridize to their target variant HTRA1 gene with the same degree of specificity.

In one embodiment, the invention provides a pair of primers that specifically detect a variation in the non-coding region of the HTRA1 gene that is correlated with the occurrence of AMD in humans, such as the variation identified as SNP rs 11200638. In such a case, the first primer hybridizes upstream from the variation and a second primer hybridizes downstream from the variation. It is understood that one of the primers hybridizes to one strand of a region of DNA that comprises a variation in the non-coding region of the HTRA1 gene that is correlated with the occurrence of AMD, and the second primer hybridizes to the complementary strand of a region of DNA that comprises a variation in the non-coding region of the HTRA1 gene that is correlated with the occurrence of AMD in humans. As used herein, the term “region of DNA” refers to a sub-chromosomal length of DNA.

In another embodiment, the invention provides an allele-specific primer that hybridizes to a site on target DNA that overlaps a variation in the non-coding region of the HTRA1 gene that is correlated with the occurrence of AMD in humans. An allele-specific primer of the invention only primes amplification of an allelic form to which the primer exhibits perfect complementarity. This primer may be used, for example, in conjunction with a second primer which hybridizes at a distal site. Amplification can thus proceed from the two primers, resulting in a detectable product that indicates the presence of a variant HTRA1 gene that is correlated with the occurrence of AMD in humans.

### 3. Detection Assays

In certain embodiments, the invention relates to polynucleotides useful for detecting a variation in the non-coding region of the HTRA1 gene that is correlated with the occurrence of age related macular degeneration, such as the variation identified as SNP rs 11200638. Preferably, these polynucleotides are capable of hybridizing under stringent hybridization conditions to a region of DNA that comprises a variation in the non-coding region, for example the promoter region of the HTRA1 gene that is correlated with the occurrence of age related macular degeneration.

The polynucleotides of the invention may be used in any assay that permits detection of a variation in the non-coding region of the human HTRA1 gene that is correlated with the occurrence of AMD. Such methods may encompass, for example, DNA sequencing, hybridization, ligation, or primer extension methods. Furthermore, any combination of these methods may be utilized in the invention. In one embodiment, the presence of a variation in the non-coding region of the human HTRA1 gene that is correlated with the occurrence of AMD is detected and/or determined by DNA sequencing. DNA sequence determination may be performed by standard methods such as dideoxy chain termination technology and gel-electrophoresis, or by other methods such as by pyrosequencing (Biotage AB, Uppsala, Sweden). For example, DNA sequencing by dideoxy chain termination may be performed using unlabeled primers and labeled (e.g., fluorescent or radioactive) terminators. Alternatively, sequencing may be performed using labeled primers and unlabeled terminators. The nucleic acid sequence of the DNA in the sample can be compared to the nucleic acid sequence of wildtype DNA to identify whether a variation in the non-coding region of the HTRA1 gene that is correlated with the occurrence of AMD is present.

In another embodiment, the presence of a variation in the non-coding region of the HTRA1 gene that is correlated with the occurrence of AMD is detected and/or determined by hybridization. In one embodiment, a polynucleotide probe hybridizes to a variation in the non-coding region of the HTRA1 gene, and flanking nucleotides, that is correlated with AMD, but not to a wildtype HTRA1 gene. The polynucleotide probe may comprise nucleotides that are fluorescently, radioactively, or chemically labeled to facilitate detection of hybridization. Hybridization may be performed and detected by standard methods known in the art, such as by Northern blotting, Southern blotting, fluorescent in situ hybridization (FISH), or by hybridization to polynucleotides immobilized on a solid support, such as a DNA array or microarray. As used herein, the term “DNA array,” and “microarray” refers to an ordered arrangement of hybridizable array elements. The array elements are arranged so that there are preferably at least one or more different array elements immobilized on a substrate surface. The hybridization signal from each of the array elements is individually distinguishable. In a preferred embodiment, the array elements comprise polynucleotides, although the present invention could also be used with cDNA or other types of nucleic acid array elements.

In a specific embodiment, the polynucleotide probe is used to hybridize genomic DNA by FISH. FISH can be used, for example, in metaphase cells, to detect a deletion in genomic DNA. Genomic DNA is denatured to separate the complimentary strands within the DNA double helix structure. The polynucleotide probe of the invention is then added to the denatured genomic DNA. If a variation in the non-coding region of the HTRA1 gene that is correlated with the occurrence of AMD is present, the probe will hybridize to the genomic DNA. The probe signal (e.g., fluorescence) can then be detected through a fluorescent microscope for the presence of absence of signal. The absence of signal, therefore, indicates the absence of a variation in the non-coding region of the HTRA1 gene that is correlated with the occurrence of AMD. An example of such a variation is a nucleotide base other than AG at position −512 relative to the putative transcription start site of the human HTRA1 gene. In another specific embodiment, a labeled polynucleotide probe is applied to immobilized polynucleotides on a DNA array. Hybridization may be detected, for example, by measuring the intensity of the labeled probe remaining on the DNA array after washing. The polynucleotides of the invention may also be used in commercial assays, such as the Taqman assay (Applied Biosystems, Foster City, Calif.).

In another embodiment, the presence of a variation in the non-coding region of the human HTRA1 gene that is correlated with the occurrence of AMD is detected and/or determined by primer extension with DNA polymerase. In one embodiment, a polynucleotide primer of the invention hybridizes immediately adjacent to the variation. A single base sequencing reaction using labeled dideoxynucleotide terminators may be used to detect the variation. The presence of a variation will result in the incorporation of the labeled terminator, whereas the absence of a variation will not result in the incorporation of the terminator. In another embodiment, a polynucleotide primer of the invention hybridizes to a variation in the non-coding region of the HTRA1 gene that is correlated with the occurrence of AMD. The primer, or a portion thereof, will not hybridize to a wildtype HTRA1 gene. The presence of a variation will result in primer extension, whereas the absence of a variation will not result in primer extension. The primers and/or nucleotides may further include fluorescent, radioactive, or chemical probes. A primer labeled by primer extension may be detected by measuring the intensity of the extension product, such as by gel electrophoresis, mass spectrometry, or any other method for detecting fluorescent, radioactive, or chemical labels.

In another embodiment, the presence of a variation in the non-coding region of the HTRA1 gene that is correlated with the occurrence of AMD is detected and/or determined by ligation. In one embodiment, a polynucleotide primer of the invention hybridizes to a variation in the non-coding region of the human HTRA1 gene that is correlated with the occurrence of AMD, such as the variation that is identified as SNP rs 11200638. The primer, or a portion thereof will not hybridize to a wildtype HTRA1 gene. A second polynucleotide that hybridizes to a region of the HTRA1 gene immediately adjacent to the first primer is also provided. One, or both, of the polynucleotide primers may be fluorescently, radioactively, or chemically labeled. Ligation of the two polynucleotide primers will occur in the presence of DNA ligase if a variation in the non-coding region of the HTRA1 gene that is correlated with the occurrence of AMD is present. Ligation may be detected by gel electrophoresis, mass spectrometry, or by measuring the intensity of fluorescent, radioactive, or chemical labels.

In another embodiment, the presence of a variation in the non-coding region of the human HTRA1 gene that is correlated with the occurrence of AMD is detected and/or determined by single-base extension (SBE). For example, a fluorescently-labeled primer that is coupled with fluorescence resonance energy transfer (FRET) between the label of the added base and the label of the primer may be used. Typically, the method, such as that described by Chen et al., (PNAS 94:10756-61 (1997), incorporated herein by reference) uses a locus-specific polynucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

Methods of detecting a variation in the non-coding region of the HTRA1 gene that is correlated with the occurrence of AMD may include amplification of a region of DNA that comprises the variation. Any method of amplification may be used. In one specific embodiment, a region of DNA comprising the variation is amplified by using polymerase chain reaction (PCR). PCR was initially described by Mullis (See e.g., U.S. Pat. Nos. 4,683,195 4,683,202, and 4,965,188, herein incorporated by reference), which describes a method for increasing the concentration of a region of DNA, in a mixture of genomic DNA, without cloning or purification. Other PCR methods may also be used for nucleic acid amplification, including but not limited to RT-PCR, quantitative PCR, real time PCR, Rapid Amplified Polymorphic DNA Analysis, Rapid Amplification of cDNA Ends (RACE), or rolling circle amplification. For example, the polynucleotide primers of the invention are combined with a DNA mixture (or any polynucleotide sequence that can be amplified with the polynucleotide primers of the invention), wherein the DNA comprises the HTRA1 gene. The mixture also includes the necessary amplification reagents (e.g., deoxyribonucleotide triphosphates, buffer, etc.) necessary for the thermal cycling reaction. According to standard PCR methods, the mixture undergoes a series of denaturation, primer annealing, and polymerase extension steps to amplify the region of DNA that comprises the variation in the non-coding region of the HTRA1 gene. An example for such a variation is the presence of a nucleotide base other than G at position −512 relative to the putative transcription start site of the human HTRA1 gene. The length of the amplified region of DNA is determined by the relative positions of the primers with respect to each other, and therefore, this length is a controllable parameter. For example, hybridization of the primers may occur such that the ends of the primers proximal to the variation are separated by 1 to 10,000 base pairs (e.g., 10 base pairs (bp) 50 bp, 200 bp, 500 bp, 1,000 bp, 2,500 bp, 5,000 bp, or 10,000 bp).

Standard instrumentation known to those skilled in the art are used for the amplification and detection of amplified DNA. For example, a wide variety of instrumentation has been developed for carrying out nucleic acid amplifications, particularly PCR, e.g. Johnson et al, U.S. Pat. No. 5,038,852 (computer-controlled thermal cycler); Wittwer et al, Nucleic Acids Research, 17: 4353-4357 (1989)(capillary tube PCR); Hallsby, U.S. Pat. No. 5,187,084 (air-based temperature control); Garner et al, Biotechniques, 14: 112-115 (1993)(high-throughput PCR in 864-well plates); Wilding et al, International application No. PCT/US93/04039 (PCR in micro-machined structures); Schnipelsky et al, European patent application No. 90301061.9 (publ. No. 0381501 A2)(disposable, single use PCR device), and the like. In certain embodiments, the invention described herein utilizes real-time PCR or other methods known in the art such as the Taqman assay.

In certain embodiments, a variant HTRA1 gene that is correlated with the occurrence of AMD in humans may be detected using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

In one embodiment, the amplified DNA is analyzed in conjunction with one of the detection methods described herein, such as by DNA sequencing. The amplified DNA may alternatively be analyzed by hybridization with a labeled probe, hybridization to a DNA array or microarray, by incorporation of biotinylated primers followed by avidin-enzyme conjugate detection, or by incorporation of ³²P-labeled deoxynucleotide triphosphates, such as dCTP or dATP, into the amplified segment. In a specific embodiment, the amplified DNA is analyzed by determining the length of the amplified DNA by electrophoresis or chromatography. For example, the amplified DNA is analyzed by gel electrophoresis. Methods of gel electrophoresis are well known in the art. See for example, Current Protocols in Molecular Biology, eds. Ausubel et al., John Wiley & Sons: 1992. The amplified DNA can be visualized, for example, by fluorescent or radioactive means, or with other dyes or markers that intercalate DNA. The DNA may also be transferred to a solid support such as a nitrocellulose membrane and subjected to Southern Blotting following gel electrophoresis. In one embodiment, the DNA is exposed to ethidium bromide and visualized under ultra-violet light.

### 4. Therapeutic Nucleic Acids Encoding SRF, AP2 Alpha, HTRA1 and CFH Polypeptides

In certain embodiments, the invention provides isolated and/or recombinant nucleic acids encoding SRF, AP2 alpha, HTRA1 and CFH polypeptides, including functional variants, disclosed herein. In certain embodiments the functional variants include dominant negative variants of SRF, AP2 alpha, HTRA1 and CFH. One skilled in the art will understand dominant negative variants to be polypeptides that compete with the wildtype polypeptides for a certain function. The utility of dominant negative variants and concepts of generating dominant negative variants are well known in the art and have been applied in many context for a long time (see for example Mendenhall M, PNAS, 85:4426-4430 (1988); Haruki N, Cancer Res. 65:3555-3561 (2005)) and some dominant negative proteins are produced commercially (for example by Cytoskeleton). In one embodiment the function that is competed for by the dominant negative variant is binding to the HTRA1 gene promoter (for example, for the transcription factors SRF or AP2 alpha). In another embodiment the function that is competed for by the dominant negative variant is the enzymatic activity of HTRA1 or CFH. In yet another embodiment the function that is competed by the dominant negative variant is the ability of HTRA1 or CFH to be secreted (see for example Mao Y, J. Bacteriol., 181:7235-7242 (1999) for dominant negative variants that inhibit protein secretion) . Other therapeutically useful variants of CFH and its general characteristics are described in patent application WO/2006/062716.

Serum response factor (SRF) is a ubiquitously expressed protein bgelonging to the MADs box family of transcription factors. SRF mediated a range of biological processes, including hematopoiesis, myogenesis and embryonic development, and may also play a role in metastatic tumor progression. SRF regulated gene transcription by either binding DNA directly or through association with cofactors (Mora-Garcia P, Stem cells, 2003; 21:123-130).

AP-2 is a sequence-specific DNA-binding protein that interacts with inducible viral and cellular enhancer elements to regulate transcription of selected genes. AP-2 factors bind and activate genes involved in a large spectrum of important biological functions including proper eye, face, body wall, limbs and neural tube development. There are three isoforms of AP-2: AP-2 alpha, beta and gamma. AP-2 alpha is the only AP-2 protein required for early morphogenesis of the lens vesicle. It binds DNA as a dimer and can form homodimers or heterodimers with other AP-2 family members.

The subject nucleic acids may be single-stranded or double stranded. Such nucleic acids may be DNA or RNA molecules. These nucleic acids may be used, for example, in methods for making SRF, AP2 alpha, HTRA1 or CFH polypeptides or as direct therapeutic agents (e.g., in a gene therapy approach).

In certain embodiments, the invention provides isolated or recombinant nucleic acid sequences that are at least 80%, 85%, 90%, 95%, 97%, 98%, 99% or 100% identical to the sequences for SRF, AP2 alpha, HTRA1, and CFH. One of ordinary skill in the art will appreciate that nucleic acid sequences complementary to the sequences for SRF, AP2 alpha, HTRA1, and CFH, and variants of the sequences for SRF, AP2 alpha, HTRA1, and CFH are also within the scope of this invention. In further embodiments, the nucleic acid sequences of the invention can be isolated, recombinant, and/or fused with a heterologous nucleotide sequence, or in a DNA library.

In other embodiments, nucleic acids of the invention also include nucleic acids that hybridize under stringent conditions to the nucleotide sequence designated in the sequences for SRF, AP2 alpha, HTRA1, and CFH, complement sequence of the sequences for SRF, AP2 alpha, HTRA1, and CFH, or fragments thereof. As discussed above, one of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from the wildtype nucleic acids for SRF, AP2 alpha, HTRA1, and CFH due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” variations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

The nucleic acids and polypeptides of the invention may be produced using standard recombinant methods. For example, the recombinant nucleic acids of the invention may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. The expression vector may also contain a selectable marker gene to allow the selection of transformed host cells. Selectable marker genes are well known in the art and will vary with the host cell used.

In certain embodiments of the invention, the subject nucleic acid is provided in an expression vector comprising a nucleotide sequence encoding SRF, AP2 alpha, HTRA1 or CFH polypeptide and operably linked to at least one regulatory sequence. Regulatory sequences are art-recognized and are selected to direct expression of SRF, AP2 alpha, HTRA1 or CFH polypeptide. Accordingly, the term regulatory sequence includes promoters, enhancers, termination sequences, preferred ribosome binding site sequences, preferred mRNA leader sequences, preferred protein processing sequences, preferred signal sequences for protein secretion, and other expression control elements. Examples of regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda , the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the invention can be produced by ligating the cloned gene, or a portion thereof, into a vector suitable for expression in either prokaryotic cells, eukaryotic cells (yeast, avian, insect or mammalian), or both. Expression vehicles for production of recombinant polypeptides include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning: A Laboratory Manual, Cold Spring Harbor Laboratory (2001). In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the 13-gal containing pBlueBac III).

In one embodiment, a vector will be designed for production of a subject SRF, AP2 alpha, HTRA1 or CFH polypeptide in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). In other embodiments, the vector is designed for production of a subject SRF, AP2 alpha, HTRA1 or CFH polypeptide in prokaryotic host cells (e.g., E. coli and B. subtilis), eukaryotic host cells such as, for example, yeast cells, insect cells, myeloma cells, fibroblast 3T3 cells, monkey kidney or COS cells, mink-lung epithelial cells, human foreskin fibroblast cells, human glioblastoma cells, and teratocarcinoma cells. Alternatively, the genes may be expressed in a cell-free system such as the rabbit reticulocyte lysate system.

As will be apparent, the subject gene constructs can be used to express the subject SRF, AP2 alpha, HTRA1 or CFH polypeptide in cells propagated in culture, e.g., to produce proteins, including fusion proteins or variant proteins, for purification.

This invention also pertains to a host cell transfected with a recombinant gene including a coding sequence for one or more of the subject SRF, AP2 alpha, HTRA1 or CFH polypeptides. The host cell may be any prokaryotic or eukaryotic cell. For example, a SRF, AP2 alpha, HTRA1 or CFH polypeptide of the invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject SRF, AP2 alpha, HTRA1 or CFH polypeptides. For example, a host cell transfected with an expression vector encoding SRF, AP2 alpha, HTRA1 or CFH polypeptide can be cultured under appropriate conditions to allow expression of the SRF, AP2 alpha, HTRA1 or CFH polypeptide to occur. SRF, AP2 alpha, HTRA1 or CFH polypeptides may be secreted and isolated from a mixture of cells and medium containing the SRF, AP2 alpha, HTRA1 or CFH polypeptides. Alternatively, the polypeptide may be retained cytoplasmically or in a membrane fraction and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a particular embodiment, the SRF, AP2 alpha, HTRA1 or CFH polypeptide is a fusion protein containing a domain which facilitates the purification of the SRF, AP2 alpha, HTRA1 or CFH polypeptide.

In another embodiment, a fusion gene coding for a purification leader sequence, such as a poly-(His)/enterokinase cleavage site sequence at the N-terminus of the desired portion of the recombinant SRF, AP2 alpha, HTRA1 or CFH polypeptide, can allow purification of the expressed fusion protein by affinity chromatography using a Ni²⁺ metal resin. The purification leader sequence can then be subsequently removed by treatment with enterokinase to provide the purified polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 5. Other Therapeutic Modalities

Antisense Polynucleotides

In certain embodiments, the invention provides polynucleotides that comprise an antisense sequence that acts through an antisense mechanism for inhibiting expression of a HTRA1 gene. Antisense technologies have been widely utilized to regulate gene expression (Buskirk et al., Chem Biol 11, 1157-63 (2004); and Weiss et al., Cell Mol Life Sci 55, 334-58 (1999)). As used herein, “antisense” technology refers to administration or in situ generation of molecules or their derivatives which specifically hybridize (e.g., bind) under cellular conditions, with the target nucleic acid of interest (mRNA and/or genomic DNA) encoding one or more of the target proteins so as to inhibit expression of that protein, e.g., by inhibiting transcription and/or translation, such as by steric hinderance, altering splicing, or inducing cleavage or other enzymatic inactivation of the transcript. The binding may be by conventional base pair complementarity, or, for example, in the case of binding to DNA duplexes, through specific interactions in the major groove of the double helix. In general, “antisense” technology refers to the range of techniques generally employed in the art, and includes any therapy that relies on specific binding to nucleic acid sequences.

A polynucleotide that comprises an antisense sequence of the present invention can be delivered, for example, as a component of an expression plasmid which, when transcribed in the cell, produces a nucleic acid sequence that is complementary to at least a unique portion of the target nucleic acid. Alternatively, the polynucleotide that comprises an antisense sequence can be generated outside of the target cell, and which, when introduced into the target cell causes inhibition of expression by hybridizing with the target nucleic acid. Polynucleotides of the invention may be modified so that they are resistant to endogenous nucleases, e.g. exonucleases and/or endonucleases, and are therefore stable in vivo. Examples of nucleic acid molecules for use in polynucleotides of the invention are phosphoramidate, phosphothioate and methylphosphonate analogs of DNA (see also U.S. Pat. Nos. 5,176,996; 5,264,564; and 5,256,775). General approaches to constructing polynucleotides useful in antisense technology have been reviewed, for example, by van der Krol et al. (1988) Biotechniques 6:958-976; and Stein et al. (1988) Cancer Res 48:2659-2668.

Antisense approaches involve the design of polynucleotides (either DNA or RNA) that are complementary to a target nucleic acid encoding HTRA1 gene. The antisense polynucleotide may bind to an mRNA transcript and prevent translation of a protein of interest. Absolute complementarity, although preferred, is not required. In the case of double-stranded antisense polynucleotides, a single strand of the duplex DNA may thus be tested, or triplex formation may be assayed. The ability to hybridize will depend on both the degree of complementarity and the length of the antisense sequence. Generally, the longer the hybridizing nucleic acid, the more base mismatches with a target nucleic acid it may contain and still form a stable duplex (or triplex, as the case may be). One skilled in the art can ascertain a tolerable degree of mismatch by use of standard procedures to determine the melting point of the hybridized complex.

Antisense polynucleotides that are complementary to the 5′ end of an mRNA target, e.g., the 5′ untranslated sequence up to and including the AUG initiation codon, should work most efficiently at inhibiting translation of the mRNA. However, sequences complementary to the 3′ untranslated sequences of mRNAs have recently been shown to be effective at inhibiting translation of mRNAs as well (Wagner, R. 1994. Nature 372:333). Therefore, antisense polynucleotides complementary to either the 5′ or 3′ untranslated, non-coding regions of a variant HTRA1 gene could be used in an antisense approach to inhibit translation of a variant HTRA1 mRNA. Antisense polynucleotides complementary to the 5′ untranslated region of an mRNA should include the complement of the AUG start codon. Antisense polynucleotides complementary to mRNA coding regions are less efficient inhibitors of translation but could also be used in accordance with the invention. Whether designed to hybridize to the 5′, 3′, or coding region of mRNA, antisense polynucleotides should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense polynucleotide to inhibit expression of HTRA1 gene. It is preferred that these studies utilize controls that distinguish between antisense gene inhibition and nonspecific biological effects of antisense polynucleotide. It is also preferred that these studies compare levels of the target RNA or protein with that of an internal control RNA or protein. Additionally, it is envisioned that results obtained using the antisense polynucleotide are compared with those obtained using a control antisense polynucleotide. It is preferred that the control antisense polynucleotide is of approximately the same length as the test antisense polynucleotide and that the nucleotide sequence of the control antisense polynucleotide differs from the antisense sequence of interest no more than is necessary to prevent specific hybridization to the target sequence.

Polynucleotides of the invention, including antisense polynucleotides, can be DNA or RNA or chimeric mixtures or derivatives or modified versions thereof, single-stranded or double-stranded. Polynucleotides of the invention can be modified at the base moiety, sugar moiety, or phosphate backbone, for example, to improve stability of the molecule, hybridization, etc. Polynucleotides of the invention may include other appended groups such as peptides (e.g., for targeting host cell receptors), or agents facilitating transport across the cell membrane (see, e.g., Letsinger et al., 1989, Proc Natl Acad Sci. USA 86:6553-6556; Lemaitre et al., 1987, Proc Natl Acad Sci. USA 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, Pharm. Res. 5:539-549 (1988)). To this end, a polynucleotide of the invention may be conjugated to another molecule, e.g., a peptide, hybridization triggered cross-linking agent, transport agent, hybridization-triggered cleavage agent, etc.

Polynucleotides of the invention, including antisense polynucleotides, may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine.

Polynucleotides of the invention may also comprise at least one modified sugar moiety selected from the group including but not limited to arabinose, 2-fluoroarabinose, xylulose, and hexose.

A polynucleotide of the invention can also contain a neutral peptide-like backbone. Such molecules are termed peptide nucleic acid (PNA)-oligomers and are described, e.g., in Perry-O'Keefe et al. (1996) Proc. Natl. Acad. Sci. USA 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, a polynucleotide of the invention comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof

In a further embodiment, polynucleotides of the invention, including antisense polynucleotides are anomeric oligonucleotides. An anomeric oligonucleotide forms specific double-stranded hybrids with complementary RNA in which, contrary to the usual units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Polynucleotides of the invention, including antisense polynucleotides, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988)), methylphosphonate oligonucleotides can be prepared by use of controlled pore glass polymer supports (Sarin et al., Proc. Natl. Acad. Sci. USA 85:7448-7451 (1988)).

While antisense sequences complementary to the coding region of an mRNA sequence can be used, those complementary to the transcribed untranslated region and to the region comprising the initiating methionine are most preferred.

Antisense polynucleotides can be delivered to cells that express target genes in vivo. A number of methods have been developed for delivering nucleic acids into cells; e.g., they can be injected directly into the tissue site, or modified nucleic acids, designed to target the desired cells (e.g., antisense polynucleotides linked to peptides or antibodies that specifically bind receptors or antigens expressed on the target cell surface) can be administered systematically.

However, it may be difficult to achieve intracellular concentrations of the antisense polynucleotides sufficient to attenuate the activity of HTRA1 gene or mRNA in certain instances. Therefore, another approach utilizes a recombinant DNA construct in which the antisense polynucleotide is placed under the control of a strong pol III or pol II promoter. The use of such a construct to transfect target cells in the patient will result in the transcription of sufficient amounts of antisense polynucleotides that will form complementary base pairs with the HTRA1 gene or mRNA and thereby attenuate the activity of HTRA1 protein. For example, a vector can be introduced in vivo such that it is taken up by a cell and directs the transcription of an antisense polynucleotide that targets HTRA1 gene or mRNA. Such a vector can remain episomal or become chromosomally integrated, as long as it can be transcribed to produce the desired antisense polynucleotide. Such vectors can be constructed by recombinant DNA technology methods standard in the art. Vectors can be plasmid, viral, or others known in the art, used for replication and expression in mammalian cells. A promoter may be operably linked to the sequence encoding the antisense polynucleotide. Expression of the sequence encoding the antisense polynucleotide can be by any promoter known in the art to act in mammalian, preferably human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionine gene (Brinster et al, Nature 296:3942 (1982)), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi Constructs—siRNAs and miRNAs

RNA interference (RNAi) is a phenomenon describing double-stranded (ds)RNA-dependent gene specific posttranscriptional silencing. Initial attempts to harness this phenomenon for experimental manipulation of mammalian cells were foiled by a robust and nonspecific antiviral defense mechanism activated in response to long dsRNA molecules (Gil et al. Apoptosis 2000, 5:107-114). The field was significantly advanced upon the demonstration that synthetic duplexes of 21 nucleotide RNAs could mediate gene specific RNAi in mammalian cells, without invoking generic antiviral defense mechanisms (Elbashir et al. Nature 2001, 411:494-498; Caplen et al. Proc Natl Acad Sci 2001, 98:9742-9747). As a result, small-interfering RNAs (siRNAs) and micro RNAs (miRNAs) have become powerful tools to dissect gene function. The chemical synthesis of small RNAs is one avenue that has produced promising results. Numerous groups have also sought the development of DNA-based vectors capable of generating such siRNA within cells. Several groups have recently attained this goal and published similar strategies that, in general, involve transcription of short hairpin (sh)RNAs that are efficiently processed to form siRNAs within cells (Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553). These reports describe methods to generate siRNAs capable of specifically targeting numerous endogenously and exogenously expressed genes.

Accordingly, the present invention provides a polynucleotide comprising an RNAi sequence that acts through an RNAi or miRNA mechanism to attenuate expression of HTRA1 gene. For instance, a polynucleotide of the invention may comprise a miRNA or siRNA sequence that attenuates or inhibits expression of HTRA1 gene. In one embodiment, the miRNA or siRNA sequence is between about 19 nucleotides and about 75 nucleotides in length, or preferably, between about 25 base pairs and about 35 base pairs in length. In certain embodiments, the polynucleotide is a hairpin loop or stem-loop that may be processed by RNAse enzymes (e.g., Drosha and Dicer).

An RNAi construct contains a nucleotide sequence that hybridizes under physiologic conditions of the cell to the nucleotide sequence of at least a portion of the mRNA transcript for HTRA1 gene. The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. It is primarily important the that RNAi construct is able to specifically target HTRA1 gene. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40mM PIPES pH 6.4, 1 mM EDTA, 50° C. or 70° C. hybridization for 12-16 hours; followed by washing).

Production of polynucleotides comprising RNAi sequences can be carried out by any of the methods for producing polynucleotides described herein. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. Polynucleotides of the invention, including wildtype or antisense polynucleotides, or those that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. Polynucleotides of the invention may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotides siRNA molecules comprise a 3′ hydroxyl group.

In other embodiments, the subject RNAi constructs are “miRNAs.” microRNAs (miRNAs) are small non-coding RNAs that direct post transcriptional regulation of gene expression through interaction with homologous mRNAs. miRNAs control the expression of genes by binding to complementary sites in target mRNAs from protein coding genes. miRNAs are similar to siRNAs. miRNAs are processed by nucleolytic cleavage from larger double-stranded precursor molecules. These precursor molecules are often hairpin structures of about 70 nucleotides in length, with 25 or more nucleotides that are base-paired in the hairpin. The RNAse III-like enzymes Drosha and Dicer (which may also be used in siRNA processing) cleave the miRNA precursor to produce an miRNA. The processed miRNA is single-stranded and incorporates into a protein complex, termed RISC or miRNP. This RNA-protein complex targets a complementary mRNA. miRNAs inhibit translation or direct cleavage of target mRNAs (Brennecke et al., Genome Biology 4:228 (2003); Kim et al., Mol. Cells 19:1-15 (2005).

In certain embodiments, miRNA and siRNA constructs can be generated by processing of longer double-stranded RNAs, for example, in the presence of the enzymes Dicer or Drosha. Dicer and Drosha are RNAse III-like nucleases that specifically cleave dsRNA. Dicer has a distinctive structure which includes a helicase domain and dual RNAse III motifs. Dicer also contains a region of homology to the RDE1/QDE2/ARGONAUTE family, which have been genetically linked to RNAi in lower eukaryotes. Indeed, activation of, or overexpression of Dicer may be sufficient in many cases to permit RNA interference in otherwise non-receptive cells, such as cultured eukaryotic cells, or mammalian (non-oocytic) cells in culture or in whole organisms. Methods and compositions employing Dicer, as well as other RNAi enzymes, are described in U.S. Pat. App. Publication No. 2004/0086884.

In one embodiment, the Drosophila in vitro system is used. In this embodiment, a polynucleotide comprising an RNAi sequence or an RNAi precursor is combined with a soluble extract derived from Drosophila embryo, thereby producing a combination. The combination is maintained under conditions in which the dsRNA is processed to RNA molecules of about 21 to about 23 nucleotides.

The miRNA and siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA and miRNA molecules. In addition, chromatography (e.g., size exclusion chromatography), glycerol gradient centrifugation, affinity purification with antibody can be used to purify siRNAs and miRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In certain embodiments, a polynucleotide of the invention that comprises an RNAi sequence or an RNAi precursor is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, (Paddison et al., Genes Dev, 2002, 16:948-58; McCaffrey et al., Nature, 2002, 418:38-9; McManus et al., RNA 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that miRNAs and siRNAs can be produced by processing a hairpin RNA in the cell.

In yet other embodiments, a plasmid is used to deliver the double-stranded RNA, e.g., as a transcriptional product. After the coding sequence is transcribed, the complementary RNA transcripts base-pair to form the double-stranded RNA.

Several RNAi constructs specifically targeting HTRA1 are commercially available (for example Stealth Select RNAi from Invitrogen).

Aptamers and Small Molecules

The present invention also provides therapeutic aptamers that specifically bind to a HTRA1 polypeptide, thereby modulating activity of the HTRA1 polypeptide. An “aptamer” may be a nucleic acid molecule, such as RNA or DNA that is capable of binding to a specific molecule with high affinity and specificity (Ellington et al., Nature 346, 818-22 (1990); and Tuerk et al., Science 249, 505-10 (1990)). An aptamer will most typically have been obtained by in vitro selection for binding of a target molecule. For example, an aptamer that specifically binds the HTRA1 polypeptide can be obtained by in vitro selection for binding to a HTRA1 polypeptide from a pool of polynucleotides. However, in vivo selection of an aptamer is also possible. Aptamers have specific binding regions which are capable of forming complexes with an intended target molecule in an environment wherein other substances in the same environment are not complexed to the nucleic acid. The specificity of the binding is defined in terms of the comparative dissociation constants (Kd) of the aptamer for its ligand (e.g., HTRA1 polypeptide) as compared to the dissociation constant of the aptamer for other materials in the environment or unrelated molecules in general. A ligand (e.g., HTRA1 polypeptide) is one which binds to the aptamer with greater affinity than to unrelated material. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less. An aptamer will typically be between about 10 and about 300 nucleotides in length. More commonly, an aptamer will be between about 30 and about 100 nucleotides in length.

Methods for selecting aptamers specific for a target of interest are known in the art. For example, organic molecules, nucleotides, amino acids, polypeptides, target features on cell surfaces, ions, metals, salts, saccharides, have all been shown to be suitable for isolating aptamers that can specifically bind to the respective ligand. For instance, organic dyes such as Hoechst 33258 have been successfully used as target ligands for in vitro aptamer selections (Werstuck and Green, Science 282:296-298 (1998)). Other small organic molecules like dopamine, theophylline, sulforhodamine B, and cellobiose have also been used as ligands in the isolation of aptamers. Aptamers have also been isolated for antibiotics such as kanamycin A, lividomycin, tobramycin, neomycin B, viomycin, chloramphenicol and streptomycin. For a review of aptamers that recognize small molecules, see (Famulok, Science 9:324-9 (1999)).

An aptamer of the invention can be comprised entirely of RNA. In other embodiments of the invention, however, the aptamer can instead be comprised entirely of DNA, or partially of DNA, or partially of other nucleotide analogs. To specifically inhibit translation in vivo, RNA aptamers are preferred. Such RNA aptamers are preferably introduced into a cell as DNA that is transcribed into the RNA aptamer. Alternatively, an RNA aptamer itself can be introduced into a cell.

Aptamers are typically developed to bind particular ligands by employing known in vivo or in vitro (most typically, in vitro) selection techniques known as SELEX (Ellington et al., Nature 346, 818-22 (1990); and Tuerk et al., Science 249, 505-10 (1990)). Methods of making aptamers are also described in, for example, (U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291).

Generally, in their most basic form, in vitro selection techniques for identifying aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques, although any means that will allow faithful, efficient amplification of selected nucleic acid sequences can be employed. The DNA pool is then in vitro transcribed to produce RNA transcripts. The RNA transcripts may then be subjected to affinity chromatography, although any protocol which will allow selection of nucleic acids based on their ability to bind specifically to another molecule (e.g., a protein or any target molecule) may be used. In the case of affinity chromatography, the transcripts are most typically passed through a column or contacted with magnetic beads or the like on which the target ligand has been immobilized. RNA molecules in the pool which bind to the ligand are retained on the column or bead, while nonbinding sequences are washed away. The RNA molecules which bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules which are capable of acting as aptamers for the target ligand. Once an aptamer sequence has been successfully identified, the aptamer may be further optimized by performing additional rounds of selection starting from a pool of oligonucleotides comprising the mutagenized aptamer sequence. For use in the present invention, the aptamer is preferably selected for ligand binding in the presence of salt concentrations and temperatures which mimic normal physiological conditions.

The unique nature of the in vitro selection process allows for the isolation of a suitable aptamer that binds a desired ligand despite a complete dearth of prior knowledge as to what type of structure might bind the desired ligand.

The association constant for the aptamer and associated ligand is preferably such that the ligand functions to bind to the aptamer and have the desired effect at the concentration of ligand obtained upon administration of the ligand. For in vivo use, for example, the association constant should be such that binding occurs well below the concentration of ligand that can be achieved in the serum or other tissue. Preferably, the required ligand concentration for in vivo use is also below that which could have undesired effects on the organism.

The present invention also provides small molecules and antibodies that specifically bind to the HTRA1 polypeptide, thereby inhibiting the activity of the HTRA1 polypeptide. In another embodiment, the small molecules and antibodies that specifically bind to the HTRA1 polypeptide prevent the secretion of HTRA1 polypeptide out of the producing cell (see Poage R, J Neurophysiol, 82:50-59 (1999) for discussion of steric hindrance through antibody binding and cross-linking of vesicles). Examples of small molecules include, without limitation, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes). An inhibitor for HTRA1 activity, NVP-LBG976, is available from Novartis, Basel (see also, Grau S, PNAS, (2005) 102: 6021-6026).

Antibodies

Another aspect of the invention pertains to antibodies. In one embodiment, an antibody that is specifically reactive with HTRA1 polypeptide may be used to detect the presence of a HTRA1 polypeptide or to inhibit activity of a HTRA1 polypeptide. For example, by using immunogens derived from the HTRA1 peptide, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of an HTRA1 peptide, an antigenic fragment which is capable of eliciting an antibody response, or a fusion protein. In a particular embodiment, the inoculated mouse does not express endogenous HTRA1, thus facilitating the isolation of antibodies that would otherwise be eliminated as anti-self antibodies. Techniques for conferring immunogenicity on a protein or peptide include conjugation to carriers or other techniques well known in the art. An immunogenic portion of a HTRA1 peptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of a HTRA1 polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with HTRA1 polypeptide and monoclonal antibodies isolated from a culture comprising such hybridoma cells.

The term “antibody” as used herein is intended to include fragments thereof which are also specifically reactive with HTRA1 polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for HTRA1 polypeptide conferred by at least one CDR region of the antibody. In preferred embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that bind specifically to HTRA1 polypeptides. For example, a method for generating a monoclonal antibody that binds specifically to HTRA1 polypeptide may comprise administering to a mouse an amount of an immunogenic composition comprising the HTRA1 polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to HTRA1 polypeptide. Once obtained, a hybridoma can be propagated in a cell culture, optionally in culture conditions where the hybridoma-derived cells produce the monoclonal antibody that binds specifically to the HTRA1 polypeptide. The monoclonal antibody may be purified from the cell culture.

Antibodies reactive to HTRA1 are commercially available (for example from Imgenex) and are also described in, for example, PCT application No. WO 00/08134.

The term “specifically reactive with” as used in reference to an antibody is intended to mean, as is generally understood in the art, that the antibody is sufficiently selective between the antigen of interest (e.g., a HTRA1 polypeptide) and other antigens that are not of interest that the antibody is useful for, at minimum, detecting the presence of the antigen of interest in a particular type of biological sample. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody-antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸10⁻⁹ or less.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the BlAcore binding assay, BlAcore AB, Uppsala, Sweden), sandwich assays (e.g., the paramagnetic bead system of IGEN International, Inc., Gaithersburg, Md.), western blots, immunoprecipitation assays, and immunohistochemistry.

In certain embodiments the present invention also provides therapeutic modalities wherein antisense polynucleotides, RNAi constructs, aptamers, small molecules, or antibody strategies, described herein, specific for HTRA1 and variants thereof, can also be combined with any or all of these aforementioned strategies, specifically designed for SRF, AP2 alpha or CFH in conjunction with HTRA1. RNAi constructs, antibodies and small molecules are available, such as RNAi constructs for SRF (Invitrogen) and AP2 alpha (OriGene Technologies) and oligonucleotides described in US Patent No. 20040109848. Antibodies for SRF and AP2 alpha are available from Abcam. An available inhibitor for SRF is distamycin A, described, for example, in (Taylor A, Mol. Cell. Biochem. 169:61-72 (1997)). Antibodies for CFH are available from USBiologicals, and siRNA constructs from OriGene and Sigma.

### 6. Pharmaceutical Compositions

The methods and compositions described herein for treating a subject suffering from AMD may be used for the prophylactic treatment of individuals who have been diagnosed or predicted to be at risk for developing AMD. In this case, the composition is administered in an amount and dose that is sufficient to delay, slow, or prevent the onset of AMD or related symptoms. Alternatively, the methods and compositions described herein may be used for the therapeutic treatment of individuals who suffer from AMD. In this case, the composition is administered in an amount and dose that is sufficient to delay or slow the progression of the condition, totally or partially, or in an amount and dose that is sufficient to reverse the condition to the point of eliminating the disorder. It is understood that an effective amount of a composition for treating a subject who has been diagnosed or predicted to be at risk for developing AMD is a dose or amount that is in sufficient quantities to treat a subject or to treat the disorder itself.

In certain embodiments, compounds of the present invention are formulated with a pharmaceutically acceptable carrier. For example, a SRF, AP2 alpha, or HTRA1 polypeptide or a nucleic acid molecule coding for a SRF, AP2 alpha, or HTRA1 polypeptide, or variant thereof, such as, for example, a dominant negative variant, can be administered alone or as a component of a pharmaceutical formulation (therapeutic composition). SRF, AP2 alpha, or HTRA1 polypeptides can also be administered in combination with a CFH polypeptide or a nucleic acid molecule coding for a CFH polypeptide, or variant thereof. The subject compounds may be formulated for administration in any convenient way for use in human medicine.

In certain embodiments, the therapeutic methods of the invention include administering the composition topically, systemically, or locally. In a specific embodiment the composition is administered locally in the eye that is affected or in risk of being affected by AMD. For example, therapeutic compositions of the invention may be formulated for administration by, for example, injection (e.g., intravenously, subcutaneously, or intramuscularly), inhalation or insufflation (either through the mouth or the nose) or oral, buccal, sublingual, transdermal, nasal, or parenteral administration. In another specific embodiment local administration can be further restricted to the area in the eye that is affected by AMD such as the area between the retinal pigment epithelium (RPE) and Bruch's membrane, for example by targeted injection of the therapeutic composition. The compositions described herein may be formulated as part of an implant or device. When administered, the therapeutic composition for use in this invention is in a pyrogen-free, physiologically acceptable form. Further, the composition may be encapsulated or injected in a viscous form for delivery to the site where the target cells are present, such as to the cells of the eye. Techniques and formulations generally may be found in Remington's Pharmaceutical Sciences, Meade Publishing Co., Easton, Pa. In addition to SRF, AP2 alpha, or HTRA1 polypeptide or a nucleic acid molecule coding for a SRF, AP2 alpha, or HTRA1 polypeptide, or variant thereof, therapeutically useful agents may optionally be included in any of the compositions as described above. Furthermore, therapeutically useful agents may, alternatively or additionally, be administered simultaneously or sequentially with SRF, AP2 alpha, or HTRA1 polypeptide or a nucleic acid molecule coding for a SRF, AP2 alpha, or HTRA1 polypeptide, or variant thereof according to the methods of the invention. In addition combinations including a CFH polypeptide or a nucleic acid molecule coding for a CFH polypeptide, or variant thereof, are contemplated.

In certain embodiments, compositions of the invention can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof

Certain compositions disclosed herein may be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the invention (e.g., an isolated or recombinantly produced nucleic acid molecule coding for SRF, AP2 alpha or HTRA1 polypeptide or an isolated or recombinantly produced SRF, AP2, HTRA1 polypeptide, or variant thereof, such as a dominant negative variant), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a subject compound, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

It is understood that the dosage regimen will be determined for an individual, taking into consideration, for example, various factors which modify the action of the subject compounds of the invention (e.g., an isolated or recombinantly produced nucleic acid molecule coding for SRF, AP2 alpha or HTRA1 polypeptide or an isolated or recombinantly produced SRF, AP2, HTRA1 polypeptide, or variant thereof, such as a dominant negative variant), the severity or stage of AMD, route of administration, and characteristics unique to the individual, such as age, weight, and size. A person of ordinary skill in the art is able to determine the required dosage to treat the subject. In one embodiment, the dosage can range from about 1.0 ng/kg to about 100 mg/kg body weight of the subject. Based upon the composition, the dose can be delivered continuously, or at periodic intervals. For example, on one or more separate occasions. Desired time intervals of multiple doses of a particular composition can be determined without undue experimentation by one skilled in the art. For example, the compound may be delivered hourly, daily, weekly, monthly, yearly (e.g. in a time release form) or as a one time delivery.

In certain embodiments, pharmaceutical compositions suitable for parenteral administration may comprise SRF, AP2 alpha or HTRA1 polypeptide or a nucleic acid molecule coding for SRF, AP2 alpha or HTRA1 polypeptide, or variant thereof, such as a dominant negative variant, in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments, the present invention also provides gene therapy for the in vivo production of HTRA1 polypeptides, or variants thereof, such as a dominant negative variant. Such therapy would achieve its therapeutic effect by introduction of HTRA1 polynucleotide sequences into cells or tissues that display deregulated HTRA1 gene expression. Delivery of HTRA1 polynucleotide sequences can be achieved using a recombinant expression vector such as a chimeric virus or a colloidal dispersion system. Targeted liposomes may also be used for the therapeutic delivery of HTRA1 polynucleotide sequences. In addition, gene therapy can be used to provide in vivo production of SRF or AP2 alpha polypeptides, or variants thereof, such as a dominant negative variant. Such therapy would achieve its therapeutic effect by introduction of SRF or AP2 alpha polynucleotide sequences into cells or tissues that display deregulated HTRA1 gene expression. In certain embodiments the invention provides a combination of gene therapy, additionally providing CFH polypeptides to cells that are deficient for normal CFH function, together with a therapy providing HTRA1, SRF, or AP2 alpha.

Various viral vectors which can be utilized for gene therapy as taught herein include adenovirus, herpes virus, vaccinia, or an RNA virus such as a retrovirus. A retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing HTRA, SRF, AP2 alpha, or CFH polynucleotide. In one preferred embodiment, the vector is targeted to cells or tissues of the eye.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for HTRA, SRF, AP2 alpha, or CFH polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

Moreover, the pharmaceutical preparation can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral packages, the pharmaceutical preparation can comprise one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinacious biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of the viral particles by cells implanted at a particular target site. Such embodiments of the present invention can be used for the delivery of an exogenously purified virus, which has been incorporated in the polymeric device, or for the delivery of viral particles produced by a cell encapsulated in the polymeric device.

A person of ordinary skill in the art is able to determine the required amount to treat the subject. It is understood that the dosage regimen will be determined for an individual, taking into consideration, for example, various factors which modify the action of the subject compounds of the invention, the severity or stage of AMD, route of administration, and characteristics unique to the individual, such as age, weight, and size. A person of ordinary skill in the art is able to determine the required dosage to treat the subject. In one embodiment, the dosage can range from about 1.0 ng/kg to about 100 mg/kg body weight of the subject. The dose can be delivered continuously, or at periodic intervals. For example, on one or more separate occasions. Desired time intervals of multiple doses of a particular composition can be determined without undue experimentation by one skilled in the art. For example, the compound may be delivered hourly, daily, weekly, monthly, yearly (e.g. in a time release form) or as a one time delivery. As used herein, the term “subject” means any individual animal capable of becoming afflicted with AMD. The subjects include, but are not limited to, human beings, primates, horses, birds, cows, pigs, dogs, cats, mice, rats, guinea pigs, ferrets, and rabbits. In the preferred embodiment, the subject is a human being.

Samples used in the methods described herein may comprise cells from the eye, ear, nose, teeth, tongue, epidermis, epithelium, blood, tears, saliva, mucus, urinary tract, urine, muscle, cartilage, skin, or any other tissue or bodily fluid from which sufficient DNA or RNA can be obtained.

The sample should be sufficiently processed to render the DNA or RNA that is present available for assaying in the methods described herein. For example, samples may be processed such that DNA from the sample is available for amplification or for hybridization to another polynucleotide. The processed samples may be crude lysates where available DNA or RNA is not purified from other cellular material. Alternatively, samples may be processed to isolate the available DNA or RNA from one or more contaminants that are present in its natural source. Samples may be processed by any means known in the art that renders DNA or RNA available for assaying in the methods described herein. Methods for processing samples may include, without limitation, mechanical, chemical, or molecular means of lysing and/or purifying cells and cell lysates. Processing methods may include, for example, ion-exchange chromatography, size exclusion chromatography, affinity chromatography, hydrophobic interaction chromatography, gel filtration chromatography, ultrafiltration, electrophoresis, and immunoaffinity purification with antibodies specific for particular epitopes of the polypeptide.

### 8. Kits

Also provided herein are kits, e.g., kits for therapeutic purposes or kits for detecting a variant HTRA1 gene in a sample from an individual. In one embodiment, a kit comprises at least one container means having disposed therein a premeasured dose of a polynucleotide probe that hybridizes, under stringent conditions, to a variation in the non-coding region of the HTRA1 gene that is correlated with the occurrence of AMD in humans. In another embodiment, a kit comprises at least one container means having disposed therein a premeasured dose of a polynucleotide primer that hybridizes, under stringent conditions, adjacent to one side of a variation in the non-coding region of the HTRA1 gene that is correlated with the occurrence of AMD in humans. In a further embodiment, a second polynucleotide primer that hybridizes, under stringent conditions, to the other side of a variation in the non-coding region of the HTRA1 gene that is correlated with the occurrence of AMD in humans is provided in a premeasured dose. Kits further comprise a label and/or instructions for the use of the therapeutic or diagnostic kit in the detection of HTRA1 in a sample. Kits may also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material). In yet another embodiment the polynucleotide probe that hybridizes, under stringent conditions, to a variation in the non-coding region of the HTRA1 gene that is correlated with the occurrence of AMD in humans is combined with a second polynucleotide probe that hybridizes, under stringent conditions, to a variation in the CFH gene that is correlated with the occurrence of AMD in humans.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Cold Spring Harbor Laboratory (2001); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And

Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### Examples

The following examples are for illustrative purposed and not intended to be limiting in any way.

The following Methods and Materials were used in the work described herein, particularly in Examples 1 and 2.

### Study Participants

Both previously (L. Baum et al., Ophthalmologica 217, 111 (2003), C. P. Pang et al., Ophthalmologica 214, 289 (2000)) and newly recruited participants were used in this study. All recruitment was carried out according to the criteria described in (C. P. Pang et al., Ophthalmologica 214, 289 (2000)). Briefly, all participants received a standard examination protocol and visual-acuity measurement. Slitlamp biomicroscopy of the fundi was performed by an experienced ophthalmologist, and stereoscopic color fundus photographs were taken by a trained ophthalmic photographer. Grading was carried out using the standard classification suggested by the International Age related Maculopathy Epidemiological Study Group. Controls showed no sign of AMD or any other major eye diseases except senile cataracts. During history taking, participants were asked about their smoking habits and that information was recorded. A smoker was defined as a person who smoked at least 5 cigarettes daily for more than one year. Smokers were subdivided into three groups: those who had never smoked, those who were ex-smokers, and those who were current smokers.

Of the 117 cases available to Applicants, Applicants excluded any classified as being at AMD stage 3 or 4 (n=18) to select only the “wet” cases of AMD. To more closely match the age distribution between cases and controls, Applicants excluded cases >90 years of age (n=2). Because the original control population (n=153) was significantly younger than the cases, Applicant excluded cases <65 years of age (n=22). The characteristics of the final group of 96 cases and 130 controls are given in Table 1.

TABLE 1
Characteristics of cases and controls in the Hong Kong cohort.

| | Cases (AMD Grade 5) | Controls |
|---|---|---|
| Total | 96 | 130 |
| Males (%) | 68 | 33 |
| Mean age (± s.d.) (years) | 74.9 ± 6.8 | 74.2 ± 5.7 |
| Age range (years) | 60-89 | 65-99 |
| Smokers (%) | 63 | 26 |

### Genotyping

Applicant genotyped each individual using the Affymetrix GeneChip Mapping 100K Set of microarrays. The SNP genotyping assay consisted of two chips (XbaI and HindIII) with 58,960 and 57,244 SNPs, respectively. Approximately 250 ng of genomic DNA was processed for each chip according to the Affymetrix protocol (H. Matsuzaki et al., Nat Methods 1, 109 (2004)).

Applicant deemed only those individual chips achieving a call rate of >90% to be usable for analysis. 268 individuals were genotyped for HindIII and 266 for HindIII and XbaI.

Individual autosomal SNP data quality was assessed by examining the call rates. SNPs with call rates <85% were eliminated from the analysis. To further eliminate SNPs with possible genotyping errors, Applicant excluded heterozygous SNPs without any observed heterozygotes and SNPs with only heterozygotes. To eliminate uninformative SNPs, applicant excluded non-heterozygous SNPs. Finally, deviations from Hardy Weinberg Equilibrium (HWE) were assessed, and Applicant excluded SNPs with a HWE X²>50. Through these exclusions, largely due to low call rates of <85%, 97,824 autosomal SNPs remained for analysis. These data are summarized in Table 2.

TABLE 2
Genotyping data quality.

| Number of Individuals | |
|---|---|
| Hind | 268 |
| Xba | 267* |
| Per-chip data quality | |
| Median call rate per chip (Hind) | 99.41% |
| Median call rate per chip (Xba) | 99.33% |
| Minimum call rate per chip (Hind) | 94.33% |
| Minimum call rate per chip (Xba) | 76.72%* |
| Per-individual data quality | |
| Average number of matches for | 30.7 |
| common SNPs between two chips^{#} | |
| Minimum number of matches for | 26 |
| common SNPs between two chips^{#} | |
| Total Number of SNPs | 116204 |
| Number of Autosomal SNPs | 113841 |
| Call rate (per-SNP) | |
| SNPs with 100% call rate | 71156 |
| SNPs with call rate between 85% and 100% | 41934 |
| SNPs with call rate less than 85% | 751 |
| SNPs with call rate above 85% | 113090 |
| (Hind; 40 or less NoCalls) | |
| Locus Polymorphism | |
| (for autosomal SNPs with call rates > 85%) | |
| Number of SNPs with no polymorphism observed | 14867 |
| Number of SNPs with only heterozygotes observed | 17 |
| Number of polymorphic SNPs with | 36 |
| no heterozygotes observed | |
| Number of SNPs with minor allele frequency < 0.01 | 6008 |
| Hardy-Weinberg Equilibrium | |
| (for polymorphic SNPs regardless of MAF) | |
| Number of SNPs with HWE X² > 50 | 346 |
| Final number of SNPs | 97824 |

*After the one Xbal chip with the low call rate was removed there were 266 samples genotyped on the Xbal chip and the minimum call rate was 95.85%.
^{#}Out of 31 SNPs that are in common between the two chips.

### Statistical Analysis

The initial analysis was carried out by constructing 2×2 tables of the allele counts and 2×3 tables of the genotype counts for each SNP in all cases and controls. Subsequently, Pearson's X² statistics were calculated and P-values computed by comparing the X² statistic to a f distribution with 1 or 2 df for the allelic and genotypic tests, respectively. SNPs yielding a P-value smaller than 5.1×10⁻⁷ (Bonferroni adjusted significance of 0.05 [0.05/97,824]) were selected for further analysis.

Two differerent methods were used, Genomic Control (GC) and Genomic Control, F-test (GCF) H. Okamoto et al., Mol Vis 12, 156 (2006) to test for the presence of admixture in the sample. The first method, GC, uses the median of the X² values for a number of unassociated SNPs (null SNPs) in the study. For the pupose of the genomic control tests, all non-significant SNPs were considered to be null SNPs. Then, the X² value was divided by the median and compared to a X² distribution to test for significance. The second method, GCF, uses the mean of the null X² values instead of the median. The individual X² values are again divided by the mean and the resulting statistic is compared to an F distribution with 1, L degrees of freedom, where L is the number of null SNPs used to compute the mean.

Odds ratios, population attributable risks, and their respective confidence intervals were calculated using standard formulae P. Armitage, G. Berry, Statistical Methods in Medical Research (Balckwell Scientific Publications, 1971). Because of the relatively high frequency of the risk allele at rs10490924 in the case/control sample (55%), the corresponding attributable risk will be overestimated, and so does not provide a good estimate of the risk in the population.

To identify the region of interest around rs 10490924, Applicant examined the region bounded by pairwise SNPs in which all four gametes were observed R. R. Hudson, N. L. Kaplan, Genetics 111, 147 (1985), subsequently referred to as the “4-gamete region.” Applicant examined the pattern of linkage disequilibrium (LD) by constructing haplotypes for the seven internal SNPs in the 4-gamete region using the SNPHAP D. Clayton. http://www-gene.cimr.cam.ac.uk/clayton/software and the PHASE M. Stephens, N. J. Smith, P. Donnelly, Am J Hum Genet 68, 978 (2001) algorithms. Both algorithms yielded the same haplotypes in similar frequencies. Once haplotypes were reconstructed, D′, a standard measure of LD, was calculated using the Haploxt program G. R. Abecasis, W. O. Cookson, Bioinformatics 16, 182 (2000). LD patterns for the combined case/control sample were then visualized using GOLD (R. Klein et al, Ophrhalmology 113, 373 (2006)). Estimated haplotype frequencies for the case and control groups combined and for each separate group are given in Table 3.

TABLE 3
Haplotype analysis of seven SNPs: rs2421019, rs2292623, rs2292625, rs10510110,
rs2280141, rs2736911, rs10490924 in the 4 gamete region.

| | T/C | A/G | A/G | T/C | T/G | T/C | T/G | All | Case | Control |
|---|---|---|---|---|---|---|---|---|---|---|
| N1 | 2 | 1 | 2 | 2 | 2 | 2 | 1 | 0.45743 | 0.61100 | 0.34024 |
| N2 | 2 | 1 | 2 | 2 | 2 | 2 | 2 | 0.17255 | 0.09849 | 0.22908 |
| N3 | 2 | 2 | 2 | 1 | 1 | 1 | 2 | 0.14944 | 0.12047 | 0.17154 |
| N4 | 1 | 1 | 2 | 1 | 1 | 2 | 2 | 0.07105 | 0.03783 | 0.09640 |
| N5 | 1 | 1 | 2 | 1 | 1 | 2 | 1 | 0.05739 | 0.05845 | 0.05659 |
| N6 | 1 | 1 | 1 | 1 | 1 | 2 | 2 | 0.03777 | 0.03009 | 0.04363 |
| N7 | 2 | 2 | 2 | 1 | 1 | 2 | 1 | 0.02841 | 0.02597 | 0.03026 |
| N8 | 2 | 2 | 2 | 1 | 1 | 2 | 2 | 0.01989 | 0.01302 | 0.02514 |
| N9 | 2 | 1 | 2 | 2 | 2 | 1 | 2 | 0.00364 | 0.00021 | 0.00626 |
| N10 | 2 | 2 | 2 | 2 | 2 | 2 | 1 | 0.00188 | 0.00431 | 0.00003 |
| N11 | 1 | 1 | 2 | 1 | 1 | 1 | 2 | 0.00041 | 0.00004 | 0.00068 |
| N12 | 1 | 1 | 1 | 1 | 1 | 2 | 1 | 0.00006 | 0.00006 | 0.00006 |
| N13 | 2 | 1 | 1 | 2 | 2 | 2 | 2 | 0.00002 | 0.00001 | 0.00003 |

Haplotype frequency estimates as determined by PHASE for the entire population “all” and for the case and control populations separately

The most probable haplotype pair for each individual was obtained from PHASE and then used to determine haplotype counts given in Table 4.

TABLE 4
Haplotype counts (and frequency) for cases and controls
determined by PHASE using the most probable haplotype
pair as the haplotype assignment for an individual.

| Haplotype | Case | Control |
|---|---|---|
| N1 | 132 (0.634) | 87 (0.335) |
| N2 | 17 (0.082) | 62 (0.239) |
| N3 | 24 (0.115) | 45 (0.173) |
| N4 | 9 (0.043) | 29 (0.112) |
| N6 | 6 (0.029) | 11 (0.042) |
| N5 | 13 (0.063) | 10 (0.038) |
| N7 | 5 (0.024) | 10 (0.038) |
| N8 | 1 (0.005) | 4 (0.015) |
| N11 | 0 | 1 (0.004) |
| N9 | 0 | 1 (0.004) |
| N10 | 1 (0.005) | 0 |

These haplotype counts were used to create the contingency table and to estimate the effect size of the risk haplotype given in Table 5.

TABLE 5
Contingency table and effect size of the risk haplotype.

| | Copies of Risk Haplotype (N1) | | | | |
|---|---|---|---|---|---|
| | 2 | 1 | 0 | OR (95% CI) | PAR (95% CI) |
| Case | 46 (0.44) | 40 (0.39) | 18 (0.17) | 10.40 (4.68-23.14) | 0.81 (0.62-0.91) |
| Control | 14 (0.11) | 59 (0.45) | 57 (0.44) | | |

For the OR and PAR, only the autosomal recessive case is considered, where cases and controls with two copies of the N1 haplotype are compared to those with zero copies.

To investigate the pattern of LD in this region, Applicant used the publicly available HapMap database, which contains information on 45 unrelated Han Chinese individuals from Beijing (CHB). Genotypes for 183 SNPs bound by the 4-gamete region were extracted from the HapMap database. Genotypes were uploaded into Haploview (J. C. Barrett, B. Fry, J. Mailer, M. J. Daly, Bioinformatics 21, 263 (2005)) to calculate LD statistics. Not all 183 of the HapMap SNPs within in this region were genotyped or passed the default quality control checks (Hardy-Weinberg P-value>0.01, minimum percentage of genotyped samples >75%, maximum of one mendelian inconsistency and a minimum allele frequency of 0.001). Haplotype blocks were identified using the parameters set forth by Gabriel et al (S. B. Gabriel et al., Science 296, 2225 (2002)), i.e., 95% confidence intervals around D′ were used to determine blocks.

Among the putative recombination sites revealed by the four-gamete test to surround the marker SNP rs10490924 (R. J. Klein et al., Science 308, 385 (2005)), five major haplotypes, N1-N5, inferred from nine SNPs (extending 63.9 kb), were identified accounting for >90% of all haplotypes in the sample. The odds ratio (OR) for two copies of the risk haplotype, N1, is 10.40, and its 95% confidence interval (CI) overlaps with that of the single SNP rs10490924, 4.68-23.14 vs. 4.83-25.69 (Table 5). LD was measured and plotted for each pair of the nine SNPs. SNP rs10490924 appears to be in LD with the upstream SNPs in PLEKHA1, but the next SNP genotyped is too far downstream (26.3 kb) to provide meaningful information about recombination/homoplasy breakpoints. The much denser sets of SNPs from the publicly available HapMap database for the Han Chinese in Beijing (CHB) population provided by international HapMap data (D. Altshuler et al., Nature 437, 1299 (2005)) did not resolve this matter, showing that rs10490924 was not in LD with either gene in the region in this population, and did not enable Applicants to uncover the disease-causing variant.

### Identification of a new SNP

Applicant resequenced the exons of the two genes flanking rs10490924, PLEKHA1 and HTRA1, as well as a portion of the 5′ upstream sequence to capture any potential promoter variants. Applicant sequenced DNA samples from cases homozygous for the rs10490924 risk allele (TT) and controls homozygous for the non-risk allele (GG). Eighty-eight samples, 50 cases and 38 controls, were immediately available for sequencing. All sequencing steps (primer design, PCR amplification, bi-directional sequencing, and mutation analysis) were carried out by Genaissance Pharmaceuticals (New Haven, Conn.).

Applicant identified 43 polymorphisms in the 22 fragments that were sequenced in this region Table 6.

TABLE 6
Polymorphisms identified through the resequencing of the PLEKHA1 and
HTRA1 genes. The AccPos for each polymorphism refers to the position in the
GenBank accession GPI_36186.1 for PLEKHA1 and BX842242.1 for HTRA1.

| | | | | Seq. Frag. | | | |
|---|---|---|---|---|---|---|---|
| Gene | Region | rs # | AccPos | 4850223 | Change | AA Change | Type |
| PLEKHA1 | intron 2 | | 26919 | | G/A | | Noncoding |
| PLEKHA1 | intron 2 | | 27167 | 4850223 | T/C | | Noncoding |
| PLEKHA1 | intron 3 | rs9988734 | 29617 | 4850224 | A/G | | Noncoding |
| PLEKHA1 | intron 5 | | 35992 | 18578027 | -/T/( ) | | Noncoding |
| PLEKHA1 | intron 6 | rs3215235 | 45061 | 4850226 | TCTAA/- | | Noncoding |
| PLEKHA1 | intron 8 | | 53481 | 18579169 | T/G | | Noncoding |
| PLEKHA1 | intron 9 | | 53755 | 18579169 | T/C | | Noncoding |
| PLEKHA1 | intron 9 | rs11200624 | 53830 | 18579169 | A/G | | Noncoding |
| PLEKHA1 | exon 10 | | 54250 | 4850228 | A/T | Tyr 268 Phe | Nonsynonomous |
| PLEKHA1 | intron 10 | rs9783213 | 54349 | 4850228 | G/A | | Noncoding |
| PLEKHA1 | intron 10 | rs2292625 | 56149 | 4850229 | G/A | | Noncoding |
| PLEKHA1 | intron 11 | | 56407 | 4850229 | C/T | | Noncoding |
| PLEKHA1 | intron 11 | rs2292626 | 56496 | 4850229 | C/T | | Noncoding |
| PLEKHA1 | intron 11 | | 58895 | 4873333 | G/A | | Noncoding |
| PLEKHA1 | exon 12 | rs1045216 | 58979 | 4873333 | G/A | Ala 320 Thr | Nonsynonomous |
| PLEKHA1 | exon 12 | | 59444 | 4873335 | A/G/T | | Synonomous |
| HTRA1 | promoter | | 58157 | 710594798 | G/T | | Noncoding |
| HTRA1 | promoter | rs11200638 | 58120 | 710594798 | A/G | | Noncoding |
| HTRA1 | promoter | | 57997 | 710594798 | C/T | | Noncoding |
| HTRA1 | promoter | | 57992 | 710594798 | C/T | | Noncoding |
| HTRA1 | promoter | rs2672598 | 57982 | 710594798 | T/C | | Noncoding |
| HTRA1 | intron 1 | | 57018 | 710648330 | C/A | | Noncoding |
| HTRA1 | intron 1 | | 56970 | 710648330 | C/T | | Noncoding |
| HTRA1 | intron 2 | | 30047 | 27864 | C/T | | Noncoding |
| HTRA1 | intron 2 | | 29931 | 27864 | A/G | | Noncoding |
| HTRA1 | intron 3 | rs2239586 | 29429 | 27866 | C/T | | Noncoding |
| HTRA1 | intron 3 | rs2239587 | 29355 | 27868 | G/A | | Noncoding |
| HTRA1 | exon 4 | | 12401 | 27868 | C/T | | Synonomous |
| HTRA1 | intron 4 | rs2672582 | 12164 | 27870 | C/T | | Noncoding |
| HTRA1 | intron 5 | | 11659 | 27870 | GTTT/- | | Noncoding |
| HTRA1 | intron 5 | rs2672583 | 11578 | 27870 | C/T | | Noncoding |
| HTRA1 | intron 5 | | 11577 | 27871 | A/G | | Noncoding |
| HTRA1 | intron 5 | | 10679 | 27871 | C/T | | Noncoding |
| HTRA1 | intron 6 | rs2672585 | 10267 | 27871 | G/A | | Noncoding |
| HTRA1 | intron 6 | | 10263 | 27873 | C/G | | Noncoding |
| HTRA1 | intron 7 | | 8846 | 27875 | C/G | | Noncoding |
| HTRA1 | Intron 7 | | 7394 | 27875 | * | | Noncoding |
| HTRA1 | Intron 7 | | 7385 | 27875 | † | | Noncoding |
| HTRA1 | Intron 7 | | 7393 | 27875 | A/T | | Noncoding |
| HTRA1 | exon 8 | rs11538140 | 7136 | 27875 | C/T | | Synonymous |
| HTRA1 | intron 8 | rs2272599 | 7069 | 27875 | G/A | | Noncoding |
| HTRA1 | intron 8 | rs2293871 | 4993 | 27877 | T/C | | Noncoding |
| HTRA1 | exon 9 | | 4744 | 27877 | C/T | | Synonomous |

*TAAATAAAA/- (SEQ ID No. 1)
†ATAAAAAAAATAAAT/- (SEQ ID No. 2)

The primer pair (excluding the M13 tail) used for the sequencing of each fragment is given in Table 7.

TABLE 7
Forward and reverse primers used for regions
resequenced in the PLEKHA1 and HTRA1 genes

| Gene | Region | Forward primer | Reverse primer |
|---|---|---|---|
| HTRA1 | promoter | CGGATGCACCAAAGATTCTCC | TTCGCGTCCTTCAAACTAATGG |
| | | (SEQ ID No. 3) | (SEQ ID No. 4) |
| HTRA1 | exon1 | AGCCGGAGCACTGCGAGGG | CGCGAAGCTCGGTTCCGAGG |
| | | (SEQ ID No. 5) | (SEQ ID No. 6) |
| HTRA1 | exon 2 | ACGTTTTTGTGGTGAACCTGAGC | GCAACAGCCACACACACCTAGC |
| | | (SEQ ID No. 7) | (SEQ ID No. 8) |
| HTRA1 | exon 3 | GCCCGATATATAAAGGAGCGATGG | AGAAGTTTTCCTGAGCCCCTTCC |
| | | (SEQ ID No. 9) | (SEQ ID No. 10) |
| HTRA1 | exon 4 | GGGATGTTAGTTGTGAGCTCAGTTCC | GCACTAGAATCCACATGGCTTGG |
| | | (SEQ ID No. 11) | (SEQ ID No. 12) |
| HTRA1 | exon 5 | CTGGGCTTCAGAGAGAAAATCTCC | ATCCGTAGGGTCATTTGCAAGC |
| | | (SEQ ID No. 13) | (SEQ ID No. 14) |
| HTRA1 | exon 6 | AGTGCCGACCTGGAGTATGTGC | GGTGAAATGTCTGTGACCTTCTGC |
| | | (SEQ ID No. 15) | (SEQ ID No. 16) |
| HTRA1 | exon 7 | GTACCCTTCTGTGGCCCTTCC | AAGGGGCCAAGGCTAATGACC |
| | | (SEQ ID No. 17) | (SEQ ID No. 18) |
| HTRA1 | exon 8 | CAGTGAACTGAGATCGTACCACTGC | AGACAGAAGGCACCCTCCTATGG |
| | | (SEQ ID No. 19) | (SEQ ID No. 20) |
| HTRAI | exon 9 | CGTGCCTGACCCACTGATGG | CCCAAGCTGGCAAGAAAAAGC |
| | | (SEQ ID No. 21) | (SEQ ID No. 22) |
| PLEKHA1 | exon 2 | ACCTTACCTAATGTTGGCAAG | GAAGACAAATCTAAAGCCTGTATAG |
| | | (SEQ ID No. 23) | (SEQ ID No. 24) |
| PLEKHA1 | exon 3 | TATTTCCCCCTTGCTTTCAGG | CCTAAACGTAGTAATCAGGTACC |
| | | (SEQ ID No. 25) | (SEQ ID No. 26) |
| PLEKHA1 | exon 4 | CTCTTACAGTTGGGAACTGCATCC | GGGGGTGCAAAATGTTATTTCC |
| | | (SEQ ID No. 27) | (SEQ ID No. 28) |
| PLEKHA1 | exon 5 | AGAAATGCTAGCCAAGTGTGG | GCTTGAGTATGAAACCTGTTGG |
| | | (SEQ ID No. 29) | (SEQ ID No. 30) |
| PLEKHA1 | exon 6 | GAACTAGTACCTGCCCGAGTAAGC | GGTGAAAAGTACATGAAGAAAGGC |
| | | (SEQ ID No. 31) | (SEQ ID No. 32) |
| PLEKHA1 | exon 7 | CAGGACTTGTGCAAAACAAGAGG | CCCCTATTTTATCTCCTGACTCTCC |
| | | (SEQ ID No. 33) | (SEQ ID No. 34) |
| PLEKHA1 | exon 8 | CTGGGTAGCTAGAGAGGGATGAGG | GTGGAATGCTGCTTTGAAGATAGG |
| | | (SEQ ID No. 35) | (SEQ ID No. 36) |
| PLEKHA1 | exon 9 | TGTGCTGGATGGTTTAAGAAGG | TGTCAAATCTGATGGCCTAACC |
| | | (SEQ ID No. 37) | (SEQ ID No. 38) |
| PLEKHA1 | exon 10 | TGGGTTTGCTAAATCAGTGC | CCCACTTCCTGAACATATAACC |
| | | (SEQ ID No. 39) | (SEQ ID No. 40) |
| PLEKHA1 | exon 11 | CATTATTGACGCCTGTTGATGG | CTTACATGATCCTGATCACACACC |
| | | (SEQ ID No. 41) | (SEQ ID No. 42) |
| PLEKHA1 | exon 12 | TGCACATTTATGCTGCATGG | CAGAGCTTGTTCAGTCACTTTGG |
| | | (SEQ ID No. 43) | (SEQ ID No. 44) |
| PLEKHA1 | exon 12 | CCTCTCGCAGCAACTCTTTGG | CCCGAATGAGAACACACAATGC |
| | | (SEQ ID No. 45) | (SEQ ID No. 46) |

### Additional Genotyping

Following the identification of rs11200638, all 270 case and control samples were genotyped for rs11200638 using the custom TaqMan SNP genotyping assay (Applied Biosystems). Genotypes were obtained for 97 cases and 126 controls.

### Mouse Real-Time PCR

Whole retinas were isolated from C57/Black6 mice aged post-natal 1 day, 7 days, 1 month, 3 months, 6 months, 9 months, and 16 months as well as from 3 month old Rdl mice. Total RNA was extracted by TRIzol (Invitrogen). Total RNA (2 ug) was reverse transcribed to cDNA using the SuperTranscript kit (Invitrogen).

The primer pair for HTRA1 was 5′-TGGGATCCGAATGATGTCGCT (Forward) (SEQ ID NO. 47) and 5′-ACAACCATGTTCAGGGTG (Reverse) (SEQ ID NO. 48) with a length of 237 bp. The annealing temperature was 58° C. The Syber Green reagent (Bio-Rad) was employed for the PCR product labeling and the iCycler (Bio-Rad) was used for performing PCR and data collection. Semiquantative PCR was done at a total reaction volume of 25 ul, including 2.5 ul of 10× High Fidelity PCR buffer (Invitrogen), 1.5 ul of MgSO4 (50mM, Invitrogen), 0.4 ul of dNTP (25 mM, Invitrogen), 0.2 ul of Taq DNA Polymerase High Fidelity (Invitrogen), 0.2 ul of primers (0.1 mM), and 0.2 ul of cDNA.

### Computational Analysis of the HTRA1 Promoter

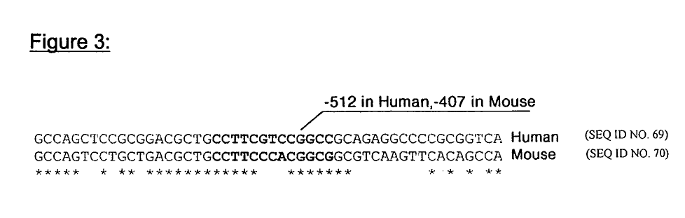

The promoter sequences for the human and mouse HTRA1 genes were obtained from the UCSC Genome Bioinformatics website (www.genome.ucsc.edu). The possible transcription factor (TF) binding sites were examined in the −2,000 to +100 by region of each promoter sequence using the positional weighting matrices extracted from the TRANSFAC databases (www.gene-regulation.com/pub/databases.html). The footprints of sequence conservation between human and mouse promoters were generated using the DnaBlockAligner program from the Wise 2.0 software package (www.ebi.ac.uk/Wise2/). Within the mouse promoter sequence, -407 was identified as the −512 G→A SNP site in humans. Only those TF binding sites that covered the SNP and were located in the human and mouse conserved promoter region were considered suitable. Results of the computational analysis are shown in FIG. 3.

Chromatin immunoprecipitation (ChIP) 1×10⁸ HeLaS3 cells were treated with formaldehyde (final concentration of 1%) for 10 min to crosslink proteins to their DNA binding targets and quenched with glycine in phosphate buffered saline (PBS) at a final concentration of 125 mM. Cells were washed twice with cold 1×PBS. The nuclear extract was prepared by swelling the cells on ice for 15 min in a hypotonic buffer (20 mM Hepes, pH 7.9, 10 mM KCl, 1 mM EDTA, pH 8, 10% glycerol, 1 mM DTT, 0.5 mM PMSF, 0.1 mM sodium orthovanadate, and protease inhibitors), followed by dounce homogenization (30 strokes). The nuclei were pelleted by brief centrifugation and lysed in radioimmunoprecipitation (RIPA) buffer (10 mM Tris-Cl, pH 8.0, 140 mM NaCl, 0.025% sodium azide, 1% Triton X-100, 0.1% SDS, 1% deoxycholic acid, 0.5 mM PMSF, 1 mM DTT, 0.1 mM sodium orthovanadate, and protease inhibitors) for 30 min on ice with repeated vortexing. The extract was sonicated with a Branson 250 Sonifier to shear the DNA (Output 20%, 100% duty cycle, five 30 second pulses) and the samples were clarified by centrifugation at 14,000 rpm at 4° C. for 15 min. 200 μL of extract was set aside for the purification of a control input DNA sample. AP-2α or SRF-DNA complexes were immunoprecipitated from the sonicated extract with an anti-AP-2α (C-18) or anti-SRF (H-300) antibody (Santa Cruz Biotechnology) overnight at 4° C. with gentle agitation. Control chromatin IP DNA was prepared using normal polyclonal rabbit IgG (Santa Cruz Biotechnology). Each immunoprecipitation sample was incubated with protein A-agarose (Upstate Biotechnology) for 1 hour at 4° C. followed by three washes with RIPA buffer and one wash with lx PBS. The antibody-protein-DNA complexes were eluted from the beads by addition of 1% SDS, 1× TE (10 mM Tris-Cl, pH 7.6, 1 mM EDTA, pH 8) and incubation at 65° C. for 10 min, followed by a second round of elution with 0.67% SDS in 1× TE, incubation for another 10 min at 65° C., and then gentle vortexing for 10 min. The beads were removed by centrifugation and the supernatants were incubated at 65° C. overnight to reverse the crosslinks. To purify the DNA, as well as the input DNA sample, RNaseA was added (200 μg/sample, in 1× TE) and then the samples were incubated at 37° C. for 2 hours, followed by an incubation with proteinase K solution (400 μg/ml proteinase K) for 2 hours at 45° C. Lastly, a phenol:chloroform:isoamyl alcohol extraction was performed and the DNA was recovered by ethanol precipitation. A more detailed description of the procedure can be found in (S. E. Hartman et al., Genes Dev 19, 2953 (2005)).

### Quantitative PCR Analysis of ChIP DNA Samples

The normal rabbit IgG, AP-2α, and SRF ChIP DNA samples were analyzed by quantitative PCR in order to test for enrichment of specific binding sites. Primers were designed to flank the candidate target region upstream of the HTRA1 gene: (−574 to −331; Forward: 5′-TCACTTCACTGTGGGTCTGG-3′ (SEQ ID No. 49); Reverse: 5′-GGGGAAAGTTCCTGCAAATC -3′) (SEQ ID No. 50). Primers were also designed to flank known AP-2α and SRF-bound human promoter regions to serve as positive controls for the ChIP PCR tests (S. Decary et al., Mol Cell Biol 22, 7877 (2002)). For AP-2α, regions upstream of insulin-like growth factor binding protein 5 (IGFBP-5; −94 to +73; Forward: 5′-CTGAGTTGGGTGTTGGGAAG-3′ (SEQ ID No. 51); Reverse: 5′-AAAGGGAAAAAGCCCACACT-3′) (SEQ ID No. 52) and E-cadherin (ECAD; −174 to −7; Forward: 5′-TAGAGGGTCACCGCGTCTATG-3′ (SEQ ID No. 53); Reverse: 5′-GGGTGCGTGGCTGCAGCCAGG-3′) (SEQ ID No. 54) were chosen (K. P. Magnusson et al., PLoS Med 3, e5 (2006)). The positive controls for SRF were upstream of Fos-related antigen 1 (FRA-1; −238 to −91; Forward: 5′-GCGGAGCTCGCAGAAACGGAGG-3′ (SEQ ID No. 55); Reverse: 5′-GGCGCTAGCCCCCTG ACGTAGCTGCCCAT-3′) (SEQ ID No. 56) (P. Adiseshaiah, S. Peddakama, Q. Zhang, D. V. Kalvakolanu, S. P. Reddy, Oncogene 24, 4193 (2005)) and early growth response protein (EGR-1; −196 to −30; Forward: 5′-CTAGGGTGCAGGATGGAGGT-3′ (SEQ ID No. 57); Reverse: 5′-GCCTCTATTTGAAGGGTCTGG-3′) (SEQ ID No. 58) (U. Philippar et al., Mol Cell 16, 867 (2004)). A negative control human promoter region, B-lymphoma and BAL-associated protein (BBAP), was also tested (−151 to +59; Forward 5′-CAGACAGCACAGGGAGGAG-3′ (SEQ ID No. 59); Reverse 5′-ACTTGTACACCCGCACGAG-3′) (SEQ ID No. 60). Quantitative PCR reactions were performed using an ABI Prism 7000 Sequence Detection System and SYBR Green Master Mix (MJ Research) and 5% DMSO. Cycling conditions were as follows: 95° C. for 5 min, 40 cycles of 95° C. for 30 sec, 52° C. for 30 sec, 72° C. for 30 sec, a final extension period of 72° C. for 10 min, followed by a 60-95° C. dissociation protocol. The ΔΔCt and fold change values were calculated relative to reference PCR reactions.

### Reporter Assay

The effect of the SNP rs11200638 on the activity of the HTRA1 promoter was assessed using a luciferase assay on transfected ARPE19 (immortalized human retinal pigment epithelium) cells from the 32nd passage and HeLaS3 cells. Constructs were designed according to the scheme in Table 8 with one contruct containing the rs11200638 wild-type allele, another containing the mutant allele and a third with no insert. Cells were grown in high glucose Dulbecco's Modified Eagle's Medium (DMEM)+10% Fetal Bovine Serum (FBS)+0.1% Gentamicin. For transfection the medium was changed to reduced serum artificial medium (Opti-MEM I) without Gentamicin. Cultures were transfected when they reached 80% confluence for the HeLaS3 cells and 50% confluence for the ARPE19 cells. Transfection was carried out with lipofectamine 2000 (2 ul/ml; Invitrogen), enhanced Green Fluorescence Protein (eGFP; 1.2 ug/ml) and the constructs (0.7 ug/ml, 1.4 ug/ml or 2.1 ug/ml for HeLaS3 and 1.4 ug/ml for ARPE19). As an additional control, each cell type was transfected with lipofectamine alone (Control). Cells were incubated with the transfection reagents for 8 hours. After removal of the transfection reagents, cells were allowed to grow for an additional 48 hours before the luciferase assay was performed. 150 ul of the lysis buffer was added to each well. 100 ul of the resulting lysate was loaded onto a black 96-well plate and 100 ul of the luciferase substrate (Bright-Glo; Promega) was added to each well. Data collection (both GFP fluorescence intensity and luciferase activity) was performed on a Packard Fusion 96-well plate reader. Independent experiments were repeated three times for each construct dose and construct type for the HeLaS3 cells and six times for each construct type for the ARPE 19 cells.

TABLE 8
Experimental design of the HTRA1 promoter reporter assay.
Information on the reporter constructs used in the assay.

| Construct name | Vector | Insert | SNP genotype |
|---|---|---|---|
| HTRA1-AA | PGL2-Basic | −834 to +119 | mutant |
| HTRA1-GG | PGL2-Basic | −834 to +119 | wild-type |
| Blank vector | PGL2-Basic | None | N/A |

| | |
|---|---|
| eGFP | GFP reporter vector for transfection efficiency control |

### Example 1

To identify novel genetic variant(s) that predispose individuals to the wet, neovascular AMD phenotype in patients of Asian descent Applicants identified 96 patients previously diagnosed with wet AMD and 130 age matched control individuals who were AMD-free (L. Baum et al., Ophthalmologica 217, 111 (2003), C. P. Pang et al., Ophthalmologica 214, 289 (2000)) from a cohort of Southeast Asians in Hong Kong. Epidemiological observations indicate that neovascular AMD is more prevalent among Asians than Caucasians (A. C. Bird, Eye 17, 457 (2003), R. Klein et al., Ophthalmology 113, 373 (2006), T. S. Chang, D. Hay, P. Courtright, Can J Ophthalmol 34, 266 (1999)), and the soft indistinct drusen that are characteristic of dry AMD are rarely seen in Asian individuals (M. A. Sandberg, A. Weiner, S. Miller, A. R. Gaudio, Ophthalmology 105, 441 (1998), M. Uyama et al., Br J Ophthalmol 84, 1018 (2000), M. Yuzawa, K. Hagita, T. Egawa, H. Minato, M. Matsui, Jpn J Ophthalmol 35, 87 (1991)). The CFH Y402H variant that occurs frequently in Caucasians (>35%) has been shown to occur less frequently in individuals of Japanese and Chinese ancestry (<5%) (N. Gotoh et al., Hum Genet 120, 139 (2006), H. Okamoto et al., Mol Vis 12, 156 (2006),M. A. Grassi et al., Hum Mutat 27, 921 (2006)). Retinal fundus photographs were examined from each of the 226 study participants. Indocyanine Green Dye (ICG) angiography was performed to exclude cases with polypoidal choroidal vasculopathy (PCV) and to verify that CNV (AMD grade 5) was present in all cases. The AMD cases and controls had a mean age of 74. Other characteristics of the study population are summarized in Table 1.

Applicant conducted a whole-genome association study on this Asian cohort to scan for single nucleotide polymorphisms (SNPs) using previously described genotyping and data quality surveillance procedures (C. P. Pang et al., Ophthalmologica 214, 289 (2000)). Of the 97,824 autosomal SNPs that were informative and passed the quality control checks, rs10490924 was the only polymorphism that showed a significant association with AMD using the Bonferroni criteria (Table 9). The allele frequency chi-square test yielded a P-value of 4.1×10⁻¹² (Table 9). The OR was 11.1 (95% confidence interval [CI] 4.83-25.69) for those carrying two copies of the risk allele when compared to wild-type homozygotes, but was indistinguishable from unity, 1.7 (95% CI 0.75-3.68), for those having a single risk allele. The risk homozygote accounted for 86% of the population attributable risk (PAR), although this number may be artificially inflated since the risk allele was carried by more than half (-55%) of the AMD cohort (Table 9). When likelihood ratio tests were adjusted for gender and smoking status or when genomic control methods were applied to control for population stratification, there was little change in significance levels.

TABLE 9
Association, odds ratios and population attributable
risk (PAR) for AMD in a Chinese population.

| | | | Odds | | Odds | |
|---|---|---|---|---|---|---|
| | Risk | Allelic χ² | ratio* | PAR* | ratio† | PAR† |
| SNP (alleles) | Allele | nominal P | (95% CI) | (95% CI) | (95% CI) | (95% CI) |
| rs10490924(G/T) | T | 4.08 × 10⁻¹² | 1.66 | 29% | 11.14 | 86% |
| | | | (0.75-3.68) | (0-63%) | (4.83-25.69) | (69%-94%) |
| rs11200638(G/A) | A | 8.24 × 10⁻¹² | 1.60 | 27% | 10.0 | 84% |
| | | | (0.71-3.61) | (0-61%) | (4.38-22.82) | (66%-93%) |

Odds ratio and PAR compare the likelihood of AMD in individuals with the listed genotype of risk allele versus those homozygous for the wild-type allele.
*Heterozygous risk individuals compared to the wild-type homozygotes.
†Homozygous risk individuals compared to the wild-type homozygotes.

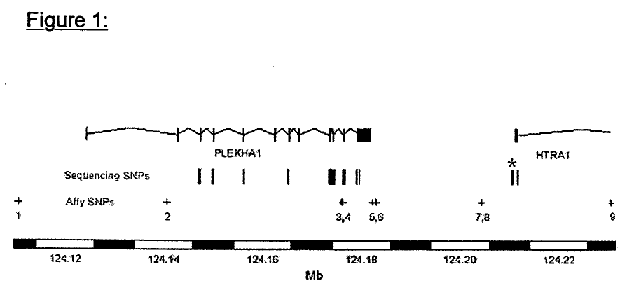

SNP rs10490924 resides between two genes on chromosome 10q26 (FIG. 1): PLEKHA1 encoding a pleckstrin homology domain-containing protein (GenBank ID 59338) and HTRA1 encoding a heat shock serine protease also known as PRSS11 (GenBank ID 5654). The low sequence homology across species in the intergenic region containing rs10490924 indicates that it is not evolutionarily conserved (FIG. 1). Chromosome 10q26 has been linked to AMD in many independent family studies and this linkage region was previously narrowed to SNP rs10490924 (P. Armitage, G. Berry, Statistical Methods in Medical Research (Balckwell Scientific Publications, 1971)). SNP rs10490924 was originally thought to result in a protein coding change in the hypothetical locus LOC387715 (A. Rivera et al., Hum Mol Genet 14, 3227 (2005), J. Jakobsdottir et al., Am J Hum Genet 77, 389 (2005)). Based on evidence of only a single cDNA sequence found in placental tissue, LOC387715 was subsequently removed from the GenBank database. Applicants hypothesized that SNP rs10490924 might be a surrogate marker that is correlated, or is in linkage disequilibrium (LD), with the putative AMD disease-causing variant in the vicinity. Haplotype analyses using Applicant's genotype data or data from the International HapMap Project were unsuccessful in identifying where the functional site resides.

Applicant therefore sequenced the entire local genomic region, including promoters, exons and intron-exon junctions of both PLEKHA1 and HTRA1, in search of the functional variant. Based on the genotypes of the marker SNP rs10490924, 50 cases that were homozygous for the risk allele and 38 controls that were homozygous for the wild-type allele were sequenced. Of the 43 SNPs or insertion/deletion polymorphisms identified (FIG. 1 and Table 6), one SNP (rs11200638), located 512 base pairs (bp) upstream of the HTRA1 putative transcriptional start site and 6,096 by downstream of SNP rs 10490924, exhibited a complete LD pattern with SNP rs10490924. Genotyping of the entire cohort revealed that SNP rs11200638 occurred at frequencies similar to those for SNP rs 10490924 (P=8.2×10⁻¹² for the allele association X² test), and the two SNPs were almost in complete LD (D′>0.99) Table 9.

### Example 2

The SNP rs11200638 is located 512 base pairs (bp) upstream of the transcription start site of the HTRA1 gene (also known as PRSS11, NM 002775).

Computational analysis of the HTRA1 promoter sequence predicted that SNP rs11200638 resides within putative binding sites for the transcription factors adaptor-related protein complex 2 alpha (AP2α and serum response factor (SRF). This DNA segment, containing the wild-type allele, is part of a CpG island and closely matches the consensus response sequences of these two transcription factors (FIG. 3). The presence of the risk allele was predicted to alter the affinity of AP2α and SRF for the HTRA1 promoter. In addition, promoter analysis with Matlnspector (Genomatix Software GmbH) suggested that the sequence variation at SNP rs11200638 might alter the binding of the Sp (Specific protein) transcription factor family member.

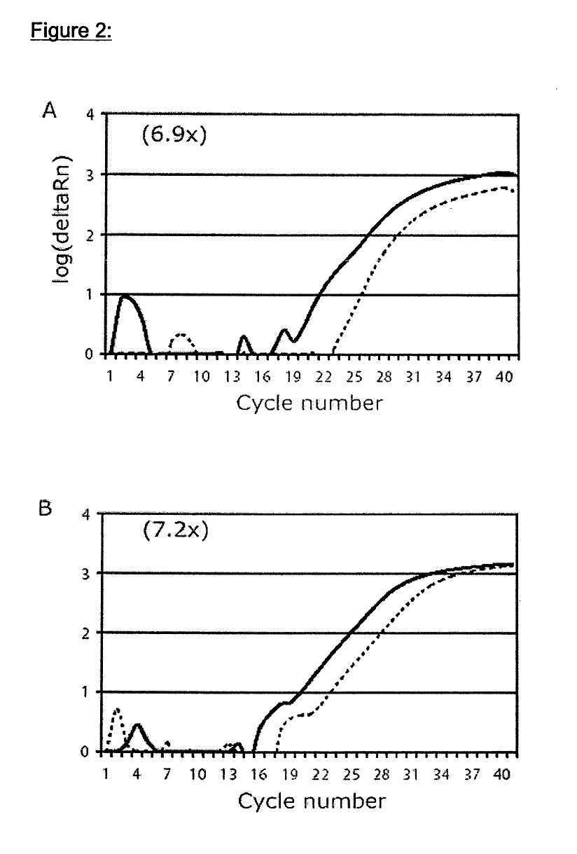

To verify that the predicted transcription factors bind to the HTRA1 promoter in cultured human cells, Applicant performed chromatin immunoprecipitation (ChIP) followed by quantitative real-time PCR analyses. Lysates were prepared from growing human cervical carcinoma cells (HeLaS3) heterozygous at rs11200638 and ChIP was conducted using rabbit polyclonal antibodies against AP2α or SRF. Quantitative PCR tests of the ChIP DNA samples confirmed that both AP2α and SRF bind upstream of the HTRA1 gene (FIGS. 2 and 3).

To investigate the influence of SNP rsl 1200638 on the HTRA1 promoter, human ARPE19 (retinal pigment epithelium) and HeLaS3 cells were transiently transfected with a luciferase reporter plasmid driven by the HTRA1 promoter harboring either the wild-type (GG) or the risk homozygote (AA) genotype. Preliminary results showed a persistent trend of higher luciferase expressions with the AA compared to the GG genotype.

The Following Methods and Materials were used in the work described herein, particularly Examples 3, 4, and 5.

### Patients

This study was approved by the University of Utah Institutional Review Board. All subjects provided informed consent prior to participation in the study. AMD patients were recruited at the Moran Eye Center (University of Utah), as were normal age-matched controls (individuals age 60 years or older with no drusen or RPE changes). All participants went through a standard examination protocol and visual acuity measurements. Slitlamp biomicroscopy of the fundi using a 90 diopter lens were performed. A pair of stereoscopic color fundus photographs)(50° were taken, centered on the fovea using a Topcon fundus camera (Topcon TRV-50VT, Topcon Optical Company, Tokyo, Japan) by trained ophthalmic photographers. Grading was carried out according to the standard grid classification system suggested by the International ARM Epidemiological Study Group for agerelated maculopathy (ARM) and AMD (A. C. Bird et al., Sury Ophthalmol 39, 367 (1995)). All abnormalities in the macula were characterized according to 1) type, size, and number of drusen, 2) RPE hyperpigmentation or hypopigmentation, and 3) advanced AMD stages including geographic atrophy (GA, dry AMD), and choroidal neovascularization (CNV, wet AMD). A total of 581 AMD patients (392 wet AMD, 189 soft confluent drusen) and 309 age and ethnicity matched normal controls participated in this study (Table 10).

TABLE 10
Characteristics of AMD Cases and
Controls Matched for Age and Ethnicity

| | Cases | Controls |
|---|---|---|
| Mean Age | 77 | 72 |
| Gender (M/F) | 291/290 | 104/205 |
| AMD (total) | 581 | 309 |
| AMD (wet) | 392 | |
| AMD (soft confluent drusen) | 189 | |

### Genotyping

The initial Utah cohort of 442 Caucasion AMD patients, including 265 wet AMD and 177 soft confluent drusen, was genotyped and allele frequencies were compared to 309 age and ethnicity matched normal controls. The expanded sample for second stage genotyping of rs10490924 and rs11200638 included 581 AMD patients (392 wet AMD, 189 soft confluent drusen).

For the rs11200638 genotype, Applicant PCR-amplified genomic DNA extracted from AMD and control patient blood samples. Oligonucleotide primers, forward 5′-ATGCCACCCACAACAACTTT-3′ (SEQ ID. No. 61) and reverse, 5′-CGCGTCCTTCAAACTAATGG-3′ (SEQ ID. No. 62) were used in PCR reactions containing 5% DMSO. DNA was denatured at 95° C.-3 minutes, followed by 35 cycles, 94° C.-30 seconds, 52° C.-30 seconds, and 72° C.-45 seconds per cycle. The PCR product was digested with Eag I to identify the G allele. For rs10490924, forward primer 5′-TACCCAGGACCGATGGTAAC-3′ (SEQ ID. No. 63) and reverse primer 5′GAGGAAGGCTGAATTGCCTA-3′ (SEQ ID. No. 64) were used for PCR amplification, PVUII digestion was used to identify the G allele. The remaining 13 SNPs were genotyped using the SNaPshot method on an ABI 3130 genetic analyzer (Applied Biosystems, Foster City, Calif.) according to the manufacturer's instructions. CFH genotyping was performed according to published methods (K. P. Magnusson et al.,. PLoS Med 3, e5 (January, 2006)).

### Data Analysis

10g26

The chi-squared test for trend for the additive model over alleles was performed to assess evidence for association. Odds ratios and 95% confidence intervals were also calculated to estimate risk size for the heterozygotes and homozygotes for the risk alleles.

Two-locus analyses (CFHY402H at 1g31 and rs11200638 at 10g26)

Two-locus analyses were performed for the CFH rs1061170 (Y402H) variant at 1 q31 and rs1120063 8 for 10g26. A contingency table based on case-control status and two-locus genotype combination was constructed. The two-locus genotype combinations across CFHY402H and rs11200638 were TT/GG, TT/AG, TT/AA, CT/GG, CT/AG, CT/AA, CC/GG, CC/AG, and CC/AA. This global, two-locus 9×2 contingency table was tested with a chi-squared statistic on 8 degrees of freedom. Odds ratios and 95% confidence intervals, comparing each genotypic combination to the baseline of homozygosity for the common allele at both loci (TT/GG), was calculated. For the risk genotypes identified, Applicant calculated population attributable risks (PAR) which indicates the proportion of total disease risk attributable to the risk genotypes, using the Levin formula (M. L. Levin, Acta Unio Int Contra Cancrum 9, 531 (1953)).

### HTRA1 Immunohistochemistry

AMD donor eyes were obtained from Utah Lions Eye Bank. Cryosections from paraformaldehydefixed eyes were incubated in 0.3% H2O02 in methanol to quench endogenous peroxidase activity. Immunohistochemistry was performed using 5 μg/ml monospecific anti-human HTRA1 polyclonal antibody (J. Chien et al., J Clin Invest 116, 1994 (July, 2006)). The VectorStain Elite ABC kit (Vector Laboratories, Burlingame, Calif.) and the VIP peroxidase substrate (Vector Laboratories) were used for HTRA1 detection and immunolabeling was captured using Nomarski optics on a Nikon Eclipse 80i microscope.

Semiquantitative RT-PCR of HT RA I mRNA in Human Lymphocyte Samples

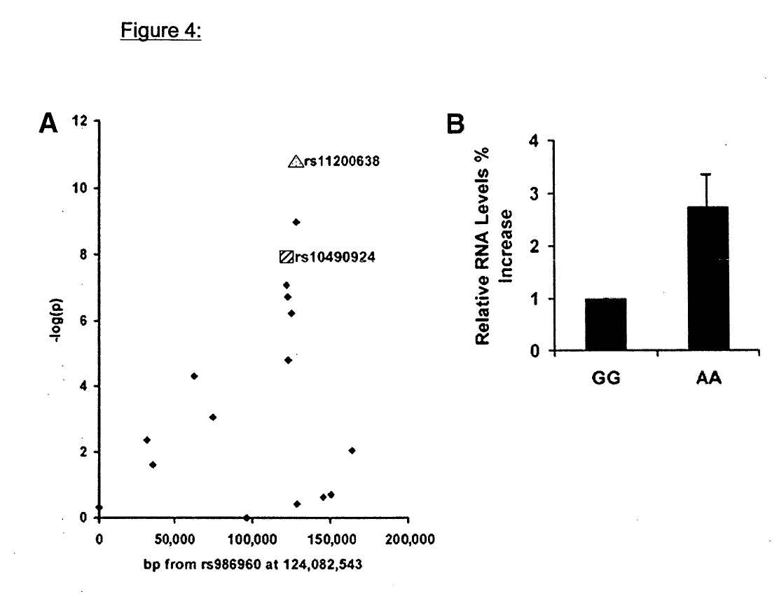

A commercial real-time PCR system (Opticon; MJ Research, Watertown, Mass.) was used for quantifying HTRAI transcript levels from patient blood lymphocyte samples. Total RNA was extracted (RNeasy; Qiagen, Valencia, Calif.) from peripheral lymphocytes of blood samples and reversetranscription PCR (RT-PCR) was performed using the QuantiTect SYBR Green RT-PCR kit (Qiagen). HTRAI primers (forward primer 5′-AGCCAAAATCAAGGATGTGG-3′ (exon 3) (SEQ ID NO. 65) and reverse primer 5′-GATGGCGACCACGAACTC-3′ (exon 4)) (SEQ ID NO. 66) and 100 nanograms (ng) total RNA from each sample were used for one step RT-PCR reactions. Standard curves were generated from 0 to 400 ng total RNA from patient lymphocyte samples. A house-keeping gene Glyseraldehyde-3-phosphate dehydrogenase (GAPDH) was amplified in parallel reactions (forward primer 5′-CTGCACCACCAACTGCTTAG 3′ (exon 7) (SEQ ID NO. 67) and reverse primer 5′-GTCTTCTGGGTGGCAGTGAT-3′) (SEQ ID NO. 68) and used to normalize HTRA1 values. HTRA1 and GAPDH standard curves showed similar amplification kinetics. Three to four patients for GG and AA genotypes were assayed in duplicate reactions and duplicate runs. Results are presented as percent increase in HTRA1 RNA levels for AA samples relative to GG samples (FIG. 4B).

### Western Analysis of HTRA1 Expression in Human RPE

25 μg of total protein of retinal pigment epithelium from four wet AMD eyes with an AA genotype and six normal eyes with a GG genotype was subject to SDS-PAGE. Western blotting was performed using 1.5 μg/ml anti-human HtrA1 polyclonal antibody (J. Chen et al., J Clin Invest 116, 1994 (Jul, 2006)). HTRA1 protein expression level was normalized to (3-actin. Statistical significance was examined using an independent samples t-test (SPSS version 13.0).

### Example 3

To identify the critical gene at the chromosome locus 10q26 in a Caucasian cohort in Utah, applicant genotyped 442 AMD cases and 309 controls, using a panel of 15 single-nucleotide polymorphisms (SNPs) centered around the highest risk associated SNP, rs 10490924 was found to have a significant association signal [P=8.1×10⁻⁸ for an additive allele-dosage model, ORₕₑₜ=1.35 (0.99, 1.86), ORₕₒₘ=6.09 (3.27, 11.34), T allele: 39.7% in cases versus 24.7% in controls]. However, of the 15 SNPs analyzed, rs11200638 was the most significantly associated variant [P=1×10⁻⁹, ORₕₑₜ=1.86 (1.35, 2.56), ORₕₒₘ=6.56 (3.23, 13.31), A allele: 40.3% in cases versus 25.2% in controls] (FIG. 4A and Table 11).

TABLE 11
Association Results for 15 SNPs at 1Og26 in AMD Cases and Controls.

| SNP | by | chi-trend | trend_p | ORhet | ORhom | chi_dom | dom_p | ORdom |
|---|---|---|---|---|---|---|---|---|
| rs986960 | 12408254 | 0.5141005 | 0.4733694 | 0.91 (0.64, 1.3 | 0.85 | 0.4099402 | 0.522 | 0.89 (0.64, 1.26) |
| rs1998345 | 12411429 | 8.1847003 | 0.0042245 | 1.37 (0.99, 1.9 | 2.02 | 6.1691034 | 0.013 | 1.48 (1.09, 2.02) |
| rs2901307 | 12411843 | 5.0544893 | 0.0245621 | 0.79 (0.55, 1.1 | 0.59 | 3.0248076 | 0.082 | 0.73 (0.52, 1.04) |
| rs4146894 | 12414537 | 16.549416 | 4.74E−05 | 1.76 (1.22, 2.5 | 2.32 | 15.445303 | 8.49E−05 | 1.96 (1.40, 2.74) |
| rs2421016 | 12415750 | 11.094448 | 0.0008661 | 1.82 (1.24, 2.6 | 2.21 | 11.989527 | 0.0005352 | 1.91 (1.32, 2.77) |
| rsl045216 | 12417918 | 0 | 1 | 0.88 (0.64, 1.2 | 1.12 | 0.2458672 | 0.62 | 0.92 (0.67, 1.26) |
| rsl049092 | 12420443 | 32.1381 | 8.14E−08 | 1.35 (0.99, 1.8 | 6.09 (3.27, 11. | 15.372106 | 8.80E−05 | 1.81 (1.35, 2.45) |
| rs3750847 | 12420541 | 27.1378 | 1.86E−07 | 1.44 (1.04, 1.9 | 5.99 (2.98, 12. | 14.608167 | 0.0001324 | 1.82 (1.34, 2.47) |
| rs3750846 | 12420555 | 18.646568 | 1.57E−05 | 1 40 (1.02, 1.9 | 4.86 (2.32, 10. | 10.332306 | 0.0013074 | 1.65 (1.22, 2.24) |
| rs2014307 | 12420762 | 24.366379 | 5.90E−07 | 0.61 (0.44, 0.8 | 0.23 | 14.867718 | 0.0001154 | 0.54 (0.39, 0.74) |
| rs1120063 | 12421052 | 37.2931 | 1.02E−09 | 1.86 (1.35, 2.5 | 6.56 (3.23, 13. | 28.22876 | 3.82E−07 | 2.21 (1.62, 3.01) |
| rs1049331 | 12421126 | 0.8081998 | 0.368653 | 0.71 (0.49, 1.0 | 0.88 | 2.4978763 | 0.114 | 0.75 (0.52, 1.07) |
| rs4752700 | 12422760 | 1.4022982 | 0.236339 | 0.65 (0.45, 0.9 | 0.85 | 4.176009 | 0.041 | 0.69 (0.49, 0.99) |
| rs2300431 | 12423280 | 1.6662006 | 0.1967684 | 0.74 (0.51, 1.0 | 0.81 | 2.4436668 | 0.118 | 0.76 (0.53, 1.07) |
| rs714816 | 12424633 | 6.812544 | 0.0090517 | 1.11 (0.80, 1.5 | 2.26 | 2.2571312 | 0.133 | 1.27 (0.93, 1.73) |

In terms of the significance of the association, the TA haplotype across rs 10490924 and rs11200638 was superior to rs10490924 (P=2.2×10⁻⁹), but inferior to rs11200638. Applicant genotyped an additional 139 AMD patients for these two variants. The results for both SNPs increased in significance (rs10490924, P=^{1.2}×10⁻⁸; rs11200638, P=1.6×10⁻¹¹), with variant rs11200638 remaining the best single variant explaining the association [ORₕₑₜ=1.90 (1.40, 2.58), ORₕₒₘ=7.51 (3.75, 15.04)].

### Example 4

Complement factor H (CFH) has been suggested to mediate drusen formation (G. S. Hageman et al., Proc Natl Acad Sci USA 102, 7227 (2005)). In Applicant's previous whole-genome association study in which the presence of large drusen was the primary phenotype under investigation, the CFH Y402H variant was shown to be a major genetic risk factor (R. J. Klein et al., Science 308, 385 (2005)). More recently, it has been reported that the highest odds ratio (OR) for CFH Y402H was seen for cases with AMD grade 4 (i.e., the presence of CGA) in comparison to AMD grade 1 controls (E. A. Postel et al., Ophthalmology 113, 1504 (2006)). An association between AMD and CFH Y402H, as well as other intronic CFH variants, has been demonstrated for more than ten different Caucasian populations (J. Mailer et al., Nat Genet 38, 1055 (2006), S. Haddad, C. A. Chen, S. L. Santangelo, J. M. Seddon, Surv Ophthalmol 51, 316 (2006), M. Li et al., Nat Genet 38, 1049 (2006), A. Thakkinstian et al., Hum Mol Genet 15, 2784 (2006)). Applicant conducted association analyses based on genotypes at both rs11200638 and the CFH rs 1061170 (Y402H) variant at chromosome 1q31. In a global two-locus analysis enumerating all nine two-locus genotype combinations, the association with AMD was significant (x²=56.56, 8 df; P=2.2×10⁻⁹). Table 12 shows the risk estimates for each two-locus genotype combination compared with the baseline of no risk genotypes (TT at CFHY402H and GG at rs11200638).

TABLE 12
Two-locus odds ratios for HTRA1 rs11200638
and CFH rs1061170. Odds ratios with 95% confidence
intervals in parentheses were calculated to compare each
genotypic combination to the baseline of homozygosity
for the common allele at both loci (TT/GG).
SNP HTRA1 rs11200638

| CFH rs1061170 | | | | |
|---|---|---|---|---|
| (Y402H) | | GG | AG | AA |
| | TT | 1.00 | 1.80 | 3.43 |
| | | | (0.93, 3.49) | (0.62, 19.00) |
| | CT | 1.07 | 2.31 | 7.31 |
| | | (0.59, 1.94) | (1.28, 4.17) | (2.68, 19.93) |
| | CC | 3.07 | 3.97 | 31.52 |
| | | (1.50, 6.27) | (1.93, 8.15) | (4.01, 247.96) |

The association of rs11200638 to AMD was significant when analyzed conditional on the presence of the CFH C risk allele (P=5.9×10⁻⁸). In particular, this conditional analysis indicates an allele-dosage effect such that homozygotes for the A risk allele of rsl 1200638 are at an increased risk [ORₕₒₙ=7.29 (3.18, 16.74)] over that of heterozygotes [ORₕₑₜ=1.83 (1.25, 2.68)] in all AMD cases, even when compared with a baseline that includes individuals who carry the risk genotypes at CFH. With an allele-dosage model, the estimated population attributable risk (PAR) for rs11200638 is 49.3%. Consistent with an additive effect, the estimated PAR from a joint model with CFH Y402H (that is, for a risk allele at either locus) is 71.4%.

### Example 5

To investigate the functional significance of SNP rs11200638 in Caucasians, Applicant used real-time reverse transcription polymerase chain reaction (RT-PCR) to study the expression levels of HTRA1 mRNA in lymphocytes of four AMD patients carrying the risk allele AA and three normal controls carrying the normal allele GG (FIG. 4B). The HTRA1 mRNA levels in lymphocytes from AMD patients with the AA genotype were higher by a factor of 2.7 than those in normal controls with the GG genotype (FIG. 4B). The mean HTRA1 protein level in RPE of four AMD donor eyes with a homozygous AA risk allele was higher by a factor of 1.7 than that of six normal controls with a homozygous GG allele. The analysis of human eye tissue was limited to four AMD donor eyes with an AA genotype out of the 60 donors for this study. The data suggest a trend toward higher expression with the risk AA allele. Immunohistochemistry experiments revealed that HTRA1 immunolabeling is present in the drusen of three AMD patients.

The HTRA1 gene encodes a member of a family of serine proteases expressed in the mouse retina and RPE (C. Oka et al., Development 131, 1041 (2004)). HTRA1 appears to regulate the degradation of extracellular matrix proteoglycans. This activity is thought to facilitate access of other degradative matrix enzymes, such as collagenases and matrix metalloproteinases, to their substrates (S. Grau et al., J Biol. Chem. 281, 6124 (2006)). Conceivably, overexpression of HTRA1 may alter the integrity of Bruch's membrane, favoring the invasion of choroid capillaries across the extracellular matrix, as occurs in wet AMD. HTRA1 also binds and inhibits transforming growth factor-β (TGF-β), an important regulator of extracellular matrix deposition and angiogenesis (Oka et al., Development 131, 1041 (2004)).

1.-12. (canceled)

13. A method of detecting, in a sample obtained from an individual, a variant HTRA1 gene that is correlated with the occurrence of age related macular degeneration in humans, comprising:
(a) combining the sample with a polynucleotide probe that hybridizes, under stringent conditions, to a variation in the non-coding regulatory region upstream of position +1 of the putative transcription start site of the human HTRA1 gene, but not to a wildtype HTRA1 gene, thereby producing a combination; and
(b) determining whether hybridization occurs,
wherein the occurrence of hybridization indicates that a variant HTRA1 gene that is correlated with the occurrence of age related macular degeneration is present in the sample.

14. The method of claim 13 further comprising
(c) comparing hybridization that occurs in the combination with hybridization in a control, wherein the control is the same as (a) and (b) except that the polynucleotide probe of the control does not bind to the variation in the non-coding regulatory region upstream of position +1 of the putative transcription start site of the human HTRA1 gene, or binds only to a wildtype HTRA1 gene, and the sample is the same type of sample as in (a) and is treated the same as the sample in (a), and
wherein the occurrence of hybridization in the combination, but not in the control, indicates that a variant HTRA1 gene that is correlated the occurrence of with age related macular degeneration is present in the sample.

15. (canceled)

16. The method of claim 13, wherein (a) is carried out on portion combination, the method further comprising:
(i) combining a second portion of the sample with a polynucleotide probe that hybridizes, under stringent conditions, to a wildtype HTRA1 gene, thereby producing a second portion combination and;
(ii) determining whether hybridization occurs in the first portion combination and in the second portion combination,
wherein the occurrence of hybridization in the first portion combination, but not in the second portion combination, indicates that a variant HTRA1 gene that is correlated with the occurrence of age related macular degeneration is present in the sample.

17. The method of claim 13, wherein the variation in the non-coding region is a nucleotide base other than a G at position −512 relative to the putative transcription start site of the human HTRA1 gene.

18. The method of claim 13, wherein the polynucleotide probe is a DNA probe.

19. A method of detecting, in a sample obtained from an individual, a variant HTRA1 gene that is correlated with the occurrence of age related macular degeneration in humans, comprising:
(a) combining the sample with a pair of polynucleotide primers, wherein the first polynucleotide primer hybridizes to one side of position −512 relative to the putative transcription start site of the promoter of the human HTRA1 gene located in the non-coding regulatory region upstream of position +1 of the putative transcription start site of the human HTRA1 gene and the second polynucleotide primer hybridizes to the other side of position −512 relative to the putative transcription start site of the promoter of the human HTRA1 gene;
(b) amplifying DNA in the sample, thereby producing amplified DNA;
(c) sequencing amplified DNA; and
(d) detecting in amplified DNA the presence of a variation of the wild-type sequence of the promoter region of the HTRA1 gene,
wherein the presence of the variation indicates that a variant HTRA1 gene that is correlated with the occurrence of age related macular degeneration in humans is detected in the sample.

20. The method of claim 13, wherein the variation in the non-coding regulatory region upstream of position +1 of the putative transcription start site of a human HTRA1 gene corresponds to SNP rs 11200638.

21. The method of claim 13, further comprising determining in the sample the presence or absence of an additional variation in a gene that is correlated with the occurrence of age related macular degeneration in humans, wherein the additional variation is other than the variation in the non-coding regulatory region upstream of position +1 of the putative transcription site of a human HTRA1 gene.

22.-23. (canceled)

24. The method of claim 21, wherein the additional variation is the presence of histidine at position 402 of the human CFH protein, corresponding to the SNP rs1061170 and/or the presence of serine at position 69 of the human protein LOC387715, corresponding to SNP rs10490924.

25. A method of identifying or aiding in identifying an individual suffering from or at risk for development or progression of age related macular degeneration, comprising assaying a sample obtained from the individual for the presence of a nucleotide base other than a G at position −512 relative to the putative transcription start site of the human HTRA1 gene, wherein the presence of a nucleotide base other than a G at position −512 of the variant HTRA1 gene indicates that the individual suffers from or is at risk for development or progression of age related macular degeneration. 26, (Currently Amended) A method of identifying or aiding in identifying an individual suffering from or at risk for development or progression of age related macular degeneration, comprising:
(a) combining a sample obtained from the individual with a polynucleotide probe that hybridizes, under stringent conditions, to a variation in the non-coding regulatory region upstream of position +1 of the putative transcription start site of the human HTRA1 gene that is correlated with the occurrence of age related macular degeneration in humans, but does not hybridize to a wild-type HTRA1 gene, thereby producing a combination;
(b) maintaining the combination conditions appropriate for hybridization to occur; and
(c) determining whether hybridization occurs,
wherein the occurrence of hybridization indicates that the individual is at risk for developing development or progression of age related macular degeneration.

27.-29. (canceled)

30. The method of claim 26, further comprising determining in the sample the presence or absence of an additional variation that is correlated with the occurrence of age related macular degeneration in humans other than the variation in the non-coding regulatory region upstream of position +1 of the putative transcription site of the human HTRA1 gene.

31. The method of claim 30 comprising determining as the additional variation the presence histidine at position 402 of the human CFH protein, corresponding to the SNP rs 1061170; or the presence of serine at position 69 of the human protein LOC387715, corresponding to SNP rs10490924.

32. A diagnostic kit for detecting a variant HTRA1 gene in a sample from an individual, comprising:
(a) at least one container means having disposed therein a polynucleotide probe that hybridizes, under stringent conditions, to a variation in the non-coding regulatory region upstream of position +1 of the putative transcription site of the human HTRA1 gene that is correlated with the occurrence of age related macular degeneration in humans; and
(b) a label and/or instructions for the use of the diagnostic kit in the detection of a variant HTRA1 gene in a sample.

33. The kit of claim 32, further comprising a second probe that detects in the sample the presence or absence of an additional variation that is correlated with the occurrence of age related macular degeneration in humans wherein the additional variation is a variation other than a variation in the non-coding regulatory region upstream of position +1 of the putative transcription site of the human HTRA1 gene.

34. The kit of claim 33, wherein the additional variation is the presence of histidine at position 402 of the human CFH protein, corresponding to the SNP rs1061170; and/or the presence of serine at position 69 of the human protein LOC387715, corresponding to SNP rs10490924.

35. A diagnostic kit for detecting a variant HTRA1 gene in a sample from an individual, comprising:
(a) at least one container means having disposed therein a polynucleotide primer that hybridizes, under stringent conditions, adjacent to one side of a variation at position −512 relative to the putative transcription start site of the promoter region of the human HTRA1 gene that is correlated with the occurrence of age related macular degeneration in humans; and
(b) a label and/or instructions for the use of the diagnostic kit in the detection of HTRA1 in a sample.

36. The diagnostic kit of claim 35, further comprising a second polynucleotide primer that hybridizes, under stringent conditions, to the other side of the variation at position −512 relative to the putative transcription start site of the promoter region of the human in the promoter region of the HTRA1 gene that is correlated with the occurrence of age related macular degeneration in humans.

37. The kit of claim 36, further comprising a second set of primers that hybridizes to either side of an additional variation that is correlated with the occurrence of age related macular degeneration in humans, other than the variation at position −512 relative to the putative transcription start site of the promoter region of the human HTRA1 gene, wherein the additional variation comprises: histidine at position 402 of the human CFH protein, corresponding to the SNP rs1061170; or serine at position 69 of the human protein LOC387715, corresponding to SNP rs 10490924.

38. The kit of claim 32, wherein the variation in the non-coding regulatory promoter region upstream of position +1 of the putative transcription start site of a human HTRA1 gene corresponds to SNP rs 11200638.

39.-55. (canceled)

56. A composition for treating a subject suffering from or at risk for age related macular degeneration, comprising:
(a) an effective amount of an inhibitor of HTRA1 activity; and
(b) a pharmaceutically acceptable carrier.

57. A method for treating a subject suffering from or at risk for age related macular degeneration, comprising administering to the subject an effective amount of the composition of claim 56.

58. The composition of claim 56, wherein the inhibitor of HTRA1 activity is antisense RNA, siRNA, miRNA, directed to HTRA1 that reduces the amount of RNA transcribed from the HTRA1 gene; an aptamer; a small molecule; an antibody that is directed to HTRA1; a dominant negative variant of HTRA1 that reduces the activity of the wildtype HTRA1 polypeptide; or an agent that inhibits the secretion of the HTRA1 polypeptide.

59. The method of claim 19, further comprising determining in the sample the presence or absence of an additional variation in a gene that is correlated with the occurrence of age related macular degeneration in humans, wherein the additional variation is other than the variation in the non-coding regulatory region upstream of position +1 of the putative transcription site of a human HTRA1 gene.

60. The method of claim 59, wherein the additional variation is the presence of histidine at position 402 of the human CFH protein, corresponding to the SNP rs1061170 and/or the presence of serine at position 69 of the human protein LOC387715, corresponding to SNP rs10490924.


SEQUENCE LISTING (70 sequences)

SEQ ID NO: 1 - DNA, 9 bp, Artificial Sequence
  Synthetic Oligonucleotide
taaataaaa

SEQ ID NO: 2 - DNA, 15 bp, Artificial Sequence
  Synthetic Oligonucleotide
ataaaaaaaataaat

SEQ ID NO: 3 - DNA, 21 bp, Artificial Sequence
  Synthetic Oligonucleotide
cggatgcaccaaagattctcc

SEQ ID NO: 4 - DNA, 22 bp, Artificial Sequence
  Synthetic Oligonucleotide
ttcgcgtccttcaaactaatgg

SEQ ID NO: 5 - DNA, 19 bp, Artificial Sequence
  Synthetic Oligonucleotide
agccggagcactgcgaggg

SEQ ID NO: 6 - DNA, 20 bp, Artificial Sequence
  Synthetic Oligonucleotide
cgcgaagctcggttccgagg

SEQ ID NO: 7 - DNA, 23 bp, Artificial Sequence
  Synthetic Oligonucleotide
acgtttttgtggtgaacctgagc

SEQ ID NO: 8 - DNA, 22 bp, Artificial Sequence
  Synthetic Oligonucleotide
gcaacagccacacacacctagc

SEQ ID NO: 9 - DNA, 24 bp, Artificial Sequence
  Synthetic Oligonucleotide
gcccgatatataaaggagcgatgg

SEQ ID NO: 10 - DNA, 23 bp, Artificial Sequence
  Synthetic Oligonucleotide
agaagttttcctgagccccttcc

SEQ ID NO: 11 - DNA, 26 bp, Artificial Sequence
  Synthetic Oligonucleotide
gggatgttagttgtgagctcagttcc

SEQ ID NO: 12 - DNA, 23 bp, Artificial Sequence
  Synthetic Oligonucleotide
gcactagaatccacatggcttgg

SEQ ID NO: 13 - DNA, 24 bp, Artificial Sequence
  Synthetic Oligonucleotide
ctgggcttcagagagaaaatctcc

SEQ ID NO: 14 - DNA, 22 bp, Artificial Sequence
  Synthetic Oligonucleotide
atccgtagggtcatttgcaagc

SEQ ID NO: 15 - DNA, 22 bp, Artificial Sequence
  Synthetic Oligonucleotide
agtgccgacctggagtatgtgc

SEQ ID NO: 16 - DNA, 24 bp, Artificial Sequence
  Synthetic Oligonucleotide
ggtgaaatgtctgtgaccttctgc

SEQ ID NO: 17 - DNA, 21 bp, Artificial Sequence
  Synthetic Oligonucleotide
gtacccttctgtggcccttcc

SEQ ID NO: 18 - DNA, 21 bp, Artificial Sequence
  Synthetic Oligonucleotide
aaggggccaaggctaatgacc

SEQ ID NO: 19 - DNA, 25 bp, Artificial Sequence
  Synthetic Oligonucleotide
cagtgaactgagatcgtaccactgc

SEQ ID NO: 20 - DNA, 23 bp, Artificial Sequence
  Synthetic Oligonucleotide
agacagaaggcaccctcctatgg

SEQ ID NO: 21 - DNA, 20 bp, Artificial Sequence
  Synthetic Oligonucleotide
cgtgcctgacccactgatgg

SEQ ID NO: 22 - DNA, 21 bp, Artificial Sequence
  Synthetic Oligonucleotide
cccaagctggcaagaaaaagc

SEQ ID NO: 23 - DNA, 21 bp, Artificial Sequence
  Synthetic Oligonucleotide
accttacctaatgttggcaag

SEQ ID NO: 24 - DNA, 25 bp, Artificial Sequence
  Synthetic Oligonucleotide
gaagacaaatctaaagcctgtatag

SEQ ID NO: 25 - DNA, 21 bp, Artificial Sequence
  Synthetic Oligonucleotide
tatttcccccttgctttcagg

SEQ ID NO: 26 - DNA, 23 bp, Artificial Sequence
  Synthetic Oligonucleotide
cctaaacgtagtaatcaggtacc

SEQ ID NO: 27 - DNA, 24 bp, Artificial Sequence
  Synthetic Oligonucleotide
ctcttacagttgggaactgcatcc

SEQ ID NO: 28 - DNA, 22 bp, Artificial Sequence
  Synthetic Oligonucleotide
gggggtgcaaaatgttatttcc

SEQ ID NO: 29 - DNA, 21 bp, Artificial Sequence
  Synthetic Oligonucleotide
agaaatgctagccaagtgtgg

SEQ ID NO: 30 - DNA, 22 bp, Artificial Sequence
  Synthetic Oligonucleotide
gcttgagtatgaaacctgttgg

SEQ ID NO: 31 - DNA, 24 bp, Artificial Sequence
  Synthetic Oligonucleotide
gaactagtacctgcccgagtaagc

SEQ ID NO: 32 - DNA, 24 bp, Artificial Sequence
  Synthetic Oligonucleotide
ggtgaaaagtacatgaagaaaggc

SEQ ID NO: 33 - DNA, 23 bp, Artificial Sequence
  Synthetic Oligonucleotide
caggacttgtgcaaaacaagagg

SEQ ID NO: 34 - DNA, 25 bp, Artificial Sequence
  Synthetic Oligonucleotide
cccctattttatctcctgactctcc

SEQ ID NO: 35 - DNA, 24 bp, Artificial Sequence
  Synthetic Oligonucleotide
ctgggtagctagagagggatgagg

SEQ ID NO: 36 - DNA, 24 bp, Artificial Sequence
  Synthetic Oligonucleotide
gtggaatgctgctttgaagatagg

SEQ ID NO: 37 - DNA, 22 bp, Artificial Sequence
  Synthetic Oligonucleotide
tgtgctggatggtttaagaagg

SEQ ID NO: 38 - DNA, 22 bp, Artificial Sequence
  Synthetic Oligonucleotide
tgtcaaatctgatggcctaacc

SEQ ID NO: 39 - DNA, 20 bp, Artificial Sequence
  Synthetic Oligonucleotide
tgggtttgctaaatcagtgc

SEQ ID NO: 40 - DNA, 22 bp, Artificial Sequence
  Synthetic Oligonucleotide
cccacttcctgaacatataacc

SEQ ID NO: 41 - DNA, 22 bp, Artificial Sequence
  Synthetic Oligonucleotide
cattattgacgcctgttgatgg

SEQ ID NO: 42 - DNA, 24 bp, Artificial Sequence
  Synthetic Oligonucleotide
cttacatgatcctgatcacacacc

SEQ ID NO: 43 - DNA, 20 bp, Artificial Sequence
  Synthetic Oligonucleotide
tgcacatttatgctgcatgg

SEQ ID NO: 44 - DNA, 23 bp, Artificial Sequence
  Synthetic Oligonucleotide
cagagcttgttcagtcactttgg

SEQ ID NO: 45 - DNA, 21 bp, Artificial Sequence
  Synthetic Oligonucleotide
cctctcgcagcaactctttgg

SEQ ID NO: 46 - DNA, 22 bp, Artificial Sequence
  Synthetic Oligonucleotide
cccgaatgagaacacacaatgc

SEQ ID NO: 47 - DNA, 21 bp, Artificial Sequence
  Synthetic Oligonucleotide
tgggatccgaatgatgtcgct

SEQ ID NO: 48 - DNA, 18 bp, Artificial Sequence
  Synthetic Oligonucleotide
acaaccatgttcagggtg

SEQ ID NO: 49 - DNA, 20 bp, Artificial Sequence
  Synthetic Oligonucleotide
tcacttcactgtgggtctgg

SEQ ID NO: 50 - DNA, 20 bp, Artificial Sequence
  Synthetic Oligonucleotide
ggggaaagttcctgcaaatc

SEQ ID NO: 51 - DNA, 20 bp, Artificial Sequence
  Synthetic Oligonucleotide
ctgagttgggtgttgggaag

SEQ ID NO: 52 - DNA, 20 bp, Artificial Sequence
  Synthetic Oligonucleotide
aaagggaaaaagcccacact

SEQ ID NO: 53 - DNA, 21 bp, Artificial Sequence
  Synthetic Oligonucleotide
tagagggtcaccgcgtctatg

SEQ ID NO: 54 - DNA, 21 bp, Artificial Sequence
  Synthetic Oligonucleotide
gggtgcgtggctgcagccagg

SEQ ID NO: 55 - DNA, 22 bp, Artificial Sequence
  Synthetic Oligonucleotide
gcggagctcgcagaaacggagg

SEQ ID NO: 56 - DNA, 29 bp, Artificial Sequence
  Synthetic Oligonucleotide
ggcgctagccccctgacgtagctgcccat

SEQ ID NO: 57 - DNA, 20 bp, Artificial Sequence
  Synthetic Oligonucleotide
ctagggtgcaggatggaggt

SEQ ID NO: 58 - DNA, 21 bp, Artificial Sequence
  Synthetic Oligonucleotide
gcctctatttgaagggtctgg

SEQ ID NO: 59 - DNA, 19 bp, Artificial Sequence
  Synthetic Oligonucleotide
cagacagcacagggaggag

SEQ ID NO: 60 - DNA, 19 bp, Artificial Sequence
  Synthetic Oligonucleotide
acttgtacacccgcacgag

SEQ ID NO: 61 - DNA, 20 bp, Artificial Sequence
  Synthetic Oligonucleotide
atgccacccacaacaacttt

SEQ ID NO: 62 - DNA, 20 bp, Artificial Sequence
  Synthetic Oligonucleotide
cgcgtccttcaaactaatgg

SEQ ID NO: 63 - DNA, 20 bp, Artificial Sequence
  Synthetic Oligonucleotide
tacccaggaccgatggtaac

SEQ ID NO: 64 - DNA, 20 bp, Artificial Sequence
  Synthetic Oligonucleotide
gaggaaggctgaattgccta

SEQ ID NO: 65 - DNA, 20 bp, Artificial Sequence
  Synthetic Oligonucleotide
agccaaaatcaaggatgtgg

SEQ ID NO: 66 - DNA, 18 bp, Artificial Sequence
  Synthetic Oligonucleotide
gatggcgaccacgaactc

SEQ ID NO: 67 - DNA, 20 bp, Artificial Sequence
  Synthetic Oligonucleotide
ctgcaccaccaactgcttag

SEQ ID NO: 68 - DNA, 20 bp, Artificial Sequence
  Synthetic Oligonucleotide
gtcttctgggtggcagtgat

SEQ ID NO: 69 - DNA, 50 bp, Homo sapiens
gccagctccgcggacgctgccttcgtccggccgcagaggccccgcggtca

SEQ ID NO: 70 - DNA, 50 bp, Mus musculus
gccagtcctgctgacgctgccttcccacggcggcgtcaagttcacagcca